(19) United States
(12) Patent Application Publication
(10) Pub. No.: US 2025/0381264 A2
(43) Pub. Date: Dec. 18, 2025

(54) HTLV-1 NUCLEIC ACID LIPID PARTICLE VACCINE

(71) Applicants: NATIONAL INSTITUTES OF BIOMEDICAL INNOVATION, HEALTH AND NUTRITION, Osaka (JP); UNIVERSITY OF THE RYUKYUS, Okinawa (JP); DAIICHI SANKYO COMPANY, LIMITED, Tokyo (JP)

(72) Inventors: Ken ISHII, Osaka (JP); Takuya FUKUSHIMA, Okinawa (JP); Yuetsu TANAKA, Okinawa (JP); Fumihiko TAKESHITA, Tokyo (JP); Makoto KOIZUMI, Tokyo (JP); Takako NIWA, Tokyo (JP); Shoko NOGUSA, Tokyo (JP); Nao JONAI, Tokyo (JP); Yoshikuni ONODERA, Tokyo (JP)

(21) Appl. No.: 18/562,059

(22) PCT Filed: May 18, 2022
(86) PCT No.: PCT/JP2022/020646

§ 371 Date: Nov. 17, 2023

(30) Foreign Application Priority Data
May 19, 2021 (JP) 2021-084942

(51) Int. Cl. A61K 39/21; A61K 9/51; A61K 39/00; A61P 31/14; A61P 37/04

(52) U.S. Cl. CPC A61K 39/21; A61K 9/5123; A61P 31/14; A61P 37/04; A61K 2039/6018

(57) ABSTRACT

Provided is a vaccine for preventing and/or treating infection with human T-cell leukemia virus type 1 (HTLV-1).

A lipid particle encapsulating a nucleic acid expressing a gp46 antigen or a Tax antigen of human T-cell leukemia virus type 1 (HTLV-1), wherein the lipid comprises a cationic lipid represented by general formula (Ia):

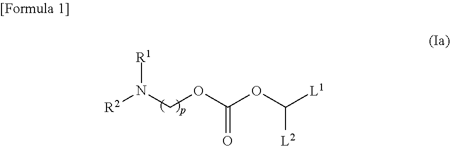

or a pharmaceutically acceptable salt thereof,
wherein
R¹ and R² each independently represent a C₁-C₃ alkyl group;
L¹ represents a C₁₇-C₁₉ alkenyl group optionally having one or more C₂-C₄ alkanoyloxy groups;
L² represents a C₁₀-C₁₉ alkyl group optionally having one or more C₂-C₄ alkanoyloxy groups, or a C₁₀-C₁₉ alkenyl group optionally having one or more C₂-C₄ alkanoyloxy groups; and
p is 3 or 4.

### TECHNICAL FIELD

The present invention relates to a nucleic-acid lipid particle vaccine encapsulating HTLV-1 mRNA.

### BACKGROUND ART

Human T-cell leukemia virus type 1 (HTLV-1) is a retrovirus having high carcinogenicity. There are 10 million to 20 million infected persons with HTLV-1 in the world and one million infected persons in Japan (Non Patent Literature 1). Of the HTLV-1 infected persons, 3 to 5% persons develop adult T cell leukemia (ATL) with a poor prognosis. At present, there are neither prophylactic vaccines for HTLV-1 infection nor drugs for preventing the onset of ATL. In addition, there is no established standard treatment contributing to a satisfactory/effective therapy for ATL. For example, if a multi-drug chemotherapy as a standard therapy, is applied, the percentage of the long-term survivors is less than 10%.

Recently, allogeneic hematopoietic stem-cell transplantation has come to be applied to ATL patients and some of the patients highly benefit from the transplantation therapy. To the antitumor effect of the allogeneic hematopoietic stem-cell transplantation, not only the effect of pre-transplantation treatments, such as high-dose anticancer drug administration and radiation of the whole body, but also an antitumor effect (graft versus leukemia effect; GVL effect) produced by donor's immunocompetent cells after the transplantation may conceivably contribute (Non Patent Literature 2). Particularly, an increase of cytotoxic T lymphocytes (CTLs) specific to a transcription activator Tax essential for HTLV-1 replication has been reported (Non Patent Literature 3). However, in the allogeneic hematopoietic stem cell transplantation, the mortality of patients due to a side effect, graft versus host disease (GVHD), is high.

Tax is a protein having an ability to activate NF-kB and being essential for the replication of HTLV-1 (Non Patent Literature 4). It has been confirmed that excessive NF-kB activation through Tax occurs in HTLV-1 infected cells and ATL cells. The NF-kB activation by Tax is clearly correlated with the canceration of infected cells (Non Patent Literature 5, Non Patent Literature 6). Non Patent Literature 7 discloses that amino acids important for NF-kB activation by Tax were identified, and that the mechanism how Tax activates NF-kB has been analyzed by use of a Tax mutant prepared by inserting a point mutation to the amino acids (Non Patent Literature 8 and Non Patent Literature 9). However, effective vaccines using Tax or a Tax mutant for preventing or treating HTLV-1 have not yet been obtained.

From the results of experiments on HTLV-1 infection using immunodeficient mice having human peripheral blood mononuclear cells (hPBMCs) transplanted therein, it was clarified that HTLV-1 infection is neutralized by an anti-gp46 antibody targeting an envelope protein gp46 of HTLV-1 (Non Patent Literature 10). Also, in transovarial transmission rat models, it is suggested that an anti-gp46 antibody having a neutralization activity can prevent transovarial transmission. However, an effective vaccine using gp46 for preventing or treating HTLV-1 has not been reported.

As an LNP preparation (LNP-mRNA) encapsulating mRNA encoding an antigen in lipid nanoparticles (LNP), a SARS-COV-2 vaccine (Non Patent Literature 11) or an influenza vaccine (patent literature 1) is known.

### CITATION LIST

### Patent Literature

Patent literature 1: International Publication No. WO2015/164674

### Non Patent Literatures

Non Patent Literature 1: Front Microbiol. (2012) 3:388.
Non Patent Literature 2: Blood (2012) 119 (10): 2409-2416.
Non Patent Literature 3: Cancer Research (2010) 70 (15): 6181-6192.
Non Patent Literature 4: Lancet Infect Dis (2007) 7:266-281.
Non Patent Literature 5: Front Microbiol. (2012) 3:406.
Non Patent Literature 6: Viruses (2011) 3 (6): 714-749.
Non Patent Literature 7: Gene Dev. (1990) 4:1875.
Non Patent Literature 8: J. Biochem. (2011) 150 (6): 679-686.
Non Patent Literature 9: J. Biol Chem. (2006) 281 (19): 13075-13082.
Non Patent Literature 10: Viruses (2016) 8 (2): 41.
Non Patent Literature 11: Science (2021) 371:1152.

### SUMMARY OF INVENTION

### Technical Problem

An object of the present invention is to provide a vaccine for preventing and/or treating infection with human T cell leukemia virus type 1 (HTLV-1).

### Solution to Problem

The present inventors have found that a nucleic-acid lipid particle encapsulating HTLV-1 mRNA (LNP-mRNA) can be applied to the treatment and prevention of the onset of adult T cell leukemia, and have arrived at the achievement of the present invention. More specifically, they have prepared LNP-mRNA-HTLV-1 encapsulating mRNA expressing HTLV-1 Tax and gp46; and administered LNP-mRNA-HTLV-1 to mice and monkeys. As a result, they have found that the production of antibodies to HTLV-1 Tax and gp46 and CTLs and the neutralization activity to inhibit the proliferation of HTLV-1 infected cells are induced in the mice and monkeys.

The gist of the present invention is as follows.

(1) A lipid particle encapsulating a nucleic acid capable of expressing a gp46 antigen or a Tax antigen of human T-cell leukemia virus type 1 (HTLV-1), wherein the lipid comprises a cationic lipid represented by general formula (Ia):

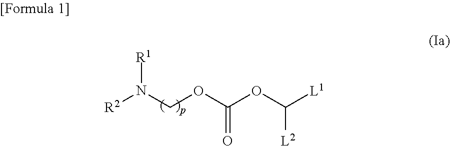

or a pharmaceutically acceptable salt thereof,
wherein
R¹ and R² each independently represent a C₁-C₃ alkyl group;
L¹ represents a C₁₇-C₁₉ alkenyl group optionally having one or more C₂-C₄ alkanoyloxy group
L² represents a C₁₀-C₁₉ alkyl group optionally having one or more C₂-C₄ alkanoyloxy groups, or a C₁₀-C₁₉ alkenyl group optionally having one or more C₂-C₄ alkanoyloxy groups; and
p is 3 or 4.

(2) The particle according to (1), wherein, in general formula (Ia), R¹ and R² both are methyl groups.

(3) The particle according to (1) or (2), wherein, in general formula (Ia), “p” is 3.

(4) The particle according to any one of (1) to (3), wherein, in general formula (Ia), L¹ is a C₁₇-C₁₉ alkenyl group optionally having one or more acetoxy group

(5) The particle according to any one of (1) to (4), wherein in general formula (Ia), L² is a C₁₀-C₁₂ alkyl group optionally having one or more acetoxy groups, or a C₁₀-C₁₉ alkenyl group optionally having one or more acetoxy groups.

(6) The particle according to any one of (1) to (4), wherein in general formula (Ia), L² is a C₁₀-C₁₂ alkyl group optionally having one or more acetoxy groups or a C₁₇-C₁₉ alkenyl group optionally having one or more acetoxy groups.

(7) The particle according to any one of (1) to (6), wherein in general formula (Ia), L¹ is an (R)-11-acetyloxy-cis-8-heptadecenyl group, a cis-8-heptadecenyl group, or an (8Z,11Z)-heptadecadienyl group.

(8) The particle according to any one of (1) to (7), wherein in general formula (Ia), L² is a decyl group, a cis-7-decenyl group, a dodecyl group, or an (R)-11-acetyloxy-cis-8-heptadecenyl group.

(9) The particle according to (1), wherein the cationic lipid is represented by the following structural formula:

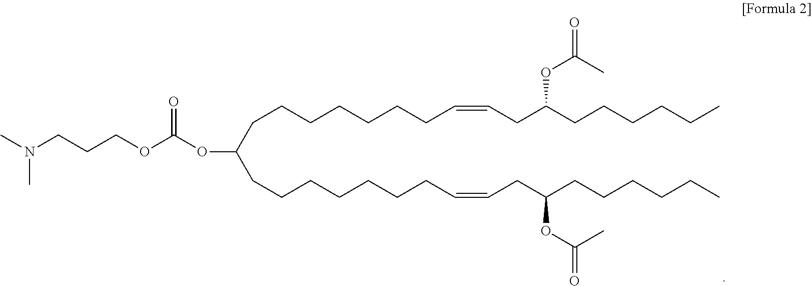 [SMILES: CCCCCC[C@H](C/C=C\CCCCCCCC(CCCCCCC/C=C\C[C@@H](CCCCCC)OC(C)=O)OC(=O)OCCCN(C)C)OC(C)=O]

(10) The particle according to (1), wherein the cationic lipid is represented by the following structural formula:

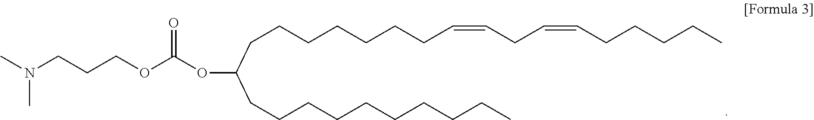 [SMILES: CCCCC/C=C\C/C=C\CCCCCCCC(CCCCCCCCCC)OC(=O)OCCCN(C)C]

(11) The particle according to (1), wherein the cationic lipid is represented by the following structural formula:

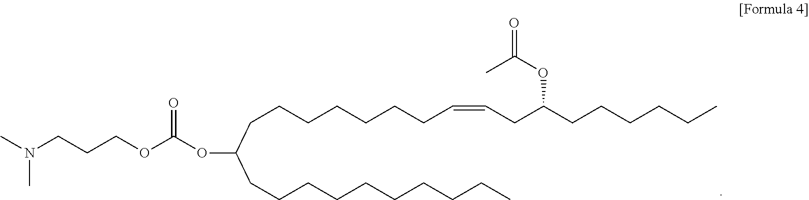 [SMILES: CCCCCCCCCCC(CCCCCCC/C=C\C[C@@H](CCCCCC)OC(C)=O)OC(=O)OCCCN(C)C]

(12) The particle according to (9) or (10), wherein the lipid further comprises an amphipathic lipid, a sterol and a PEG lipid.

(13) The particle according to (11), wherein the lipid further comprises an amphipathic lipid, a sterol and a PEG lipid.

(14) The particle according to (12), wherein the amphipathic lipid is at least one selected from the group consisting of distearoylphosphatidylcholine, dioleoylphosphatidylcholine and dioleoyl phosphatidylethanolamine.

(15) The particle according to (13), wherein the amphipathic lipid is at least one selected from the group consisting of distearoylphosphatidylcholine, dioleoylphosphatidylcholine and dioleoyl phosphatidylethanolamine.

(16) The particle according to (12) or (14), wherein the sterol is cholesterol.

(17) The particle according to (13) or (15), wherein the sterol is cholesterol.

(18) The particle according to any one of (12), (14) and (16), wherein the PEG lipid is 1,2-dimyristoyl-sn-glycerol methoxypolyethylene glycol and/or N-[methoxypoly(ethylene glycol)2000]carbamoyl]-1,2-dimyristyloxypropyl-3-amine.

(19) The particle according to any one of (13), (15) and (17), wherein the PEG lipid is 1,2-dimyristoyl-sn-glycerol methoxypolyethylene glycol and/or N-[methoxypoly(ethylene glycol)2000]carbamoyl]-1,2-dimyristyloxypropyl-3-amine.

(20) The particle according to any one of (12) to (19), wherein the lipid composition of the amphipathic lipid, the sterol, the cationic lipid, and the PEG lipid is as follows: 5 to 25% of the amphipathic lipid, 10 to 55% of the sterol, 40 to 65% of the cationic lipid, and 1 to 5% of the PEG lipid on a molar basis.

(21) The particle according to (20), wherein the amphipathic lipid is contained in a ratio of 10 to 25%.

(22) The particle according to any one of (12), (14), (16) and (18), wherein the lipid composition of the amphipathic lipid, the sterol, the cationic lipid, and the PEG lipid is as follows: 5 to 15% of the amphipathic lipid, 35 to 50% of the sterol, 40 to 55% of the cationic lipid, and 1 to 3% of the PEG lipid on a molar basis.

(23) The particle according to (22), wherein the amphipathic lipid, sterol, cationic lipid and PEG lipid are contained in a ratio of 10 to 15%, 35 to 45%, 40 to 50%, and 1 to 2.5%, respectively.

(24) The particle according to (23), wherein the PEG lipid is contained in a ratio of 1 to 2%.

(25) The particle according to any one of (13), (15), (17) and (19), wherein the lipid composition of the amphipathic lipid, the sterol, the cationic lipid, and the PEG lipid is as follows: 10 to 25% of the amphipathic lipid, 10 to 50% of the sterol, 40 to 65% of the cationic lipid, and 1 to 3% of the PEG lipid on a molar basis.

(26) The particle according to (25), wherein the sterol, cationic lipid and PEG lipid are contained in a ratio of 10 to 45%, 42.5 to 65%, and 1 to 2.5%, respectively.

(27) The particle according to (26), wherein the PEG lipid is contained in a ratio of 1 to 2%.

(28) The particle according to any one of (20) to (27), wherein a weight ratio of the total lipid to the nucleic acid is from 15 to 30.

(29) The particle according to (28), wherein the weight ratio of the total lipid to the nucleic acid is from 15 to 25.

(30) The particle according to (29), wherein the weight ratio of the total lipid to the nucleic acid is from 17.5 to 22.5.

(31) The particle according to any one of (1) to (30), wherein the gp46 antigen of human T-cell leukemia virus type 1 (HTLV-1) is a fusion protein with an oligomerization domain.

(32) The particle according to (31), wherein the oligomerization domain is fibritin.

(33) The particle according to (31) or (32), wherein the gp46 antigen of human T-cell leukemia virus type 1 (HTLV-1) consists of an amino acid sequence having an identity of at least 95% with the amino acid sequence of SEQ ID NO: 14.

(34) The particle according to any one of (1) to (33), wherein the Tax antigen of human T-cell leukemia virus type 1 (HTLV-1) has at least one mutation selected from the group consisting of T130A, L131S, L319R and L320S relative to the amino acid sequence of SEQ ID NO: 11, and the amino acid sequence consisting of amino acids except the mutant amino acids has an identity of at least 95% with the amino acid sequence of SEQ ID NO: 11.

(35) The particle according to (34), wherein the Tax antigen of human T-cell leukemia virus type 1 (HTLV-1) has mutations of T130A, L131S, L319R and L320S relative to the amino acid sequence of SEQ ID NO: 11, and the amino acid sequence consisting of amino acids except the mutant amino acids has an identity of at least 95% with the amino acid sequence of SEQ ID NO: 11.

(36) The particle according to any one of (1) to (35), wherein the Tax antigen of human T-cell leukemia virus type 1 (HTLV-1) is a fusion protein with a signal peptide.

(37) The particle according to (36), wherein the signal peptide is a peptide consisting of the sequence from positions 1 to 18 in the amino acid sequence of SEQ ID NO: 13.

(38) The particle according to any one of (31) to (37), wherein nucleic acid capable of expressing the gp46 antigen or tax antigen of human T-cell leukemia virus type 1 (HTLV-1) is mRNA comprising a cap structure (Cap), a 5′-noncoding region (5′-UTR), a coding region of the gp46 antigen or Tax antigen, a 3′-noncoding region (3′-UTR) and poly A tail (polyA).

(39) The particle according to (38), wherein the sequence of the nucleic acid capable of expressing the gp46 antigen of human T-cell leukemia virus type 1 (HTLV-1) consists of a nucleotide sequence having an identity of at least 90% with the sequence of SEQ ID NO: 17 or 18.

(40) The particle according to (38), wherein the sequence of the nucleic acid capable of expressing the Tax antigen of human T-cell leukemia virus type 1 (HTLV-1) consists of a nucleotide sequence having an identity of at least 90% with the sequence of SEQ ID NO: 20.

(41) The particle according to any one of (1) to (40), wherein the nucleic acid comprises at least one modified nucleotide.

(42) The particle according to (41), wherein the modified nucleotide comprises at least one of a pyrimidine nucleotide substituted at position 5 and/or pseudouridine optionally substituted at position 1.

(43) The particle according to (41), wherein the modified nucleotide comprises at least one selected from the group consisting of 5-methylcytidine, 5-methoxyuridine, 5-methyluridine, pseudouridine, and a 1-alkylpseudouridine.

(44) The particle according to (41), wherein the modified nucleotide comprises at least one selected from the group consisting of 5-methylcytidine, 5-methyluridine, and 1-methylpseudouridine.

(45) The particle according to any one of (1) to (44), wherein the particle has an average particle size of 30 to 300 nm.

(46) Use of the particle according to any one of (1) to (45), for preparing a composition for preventing and/or treating infection with human T-cell leukemia virus type 1 (HTLV-1).

(47) A composition comprising the particle according to any one of (1) to (45).

(48) The composition according to (47) for expressing the gp46 antigen or Tax antigen of human T-cell leukemia virus type 1 (HTLV-1) in vivo or in vitro.

(49) The composition according to (47) or (48) for use as a medicament.

(50) The composition according to (49), for inducing an immune response to human T-cell leukemia virus type 1 (HTLV-1).

(51) The composition according to (49) or (50), for preventing and/or treating infection with human T-cell leukemia virus type 1 (HTLV-1).

(52) The composition according to (49) or (50), for preventing the onset of and/or treating a disease caused by HTLV-1 selected from the group consisting of adult T cell leukemia/lymphoma (ATLL), HTLV-1 associated myelopathy (HAM) and HTLV-1 uveitis (HU), in an HTLV-1 infected person.

(53) A method for expressing the gp46 antigen or Tax antigen of human T-cell leukemia virus type 1 (HTLV-1) in vitro, comprising introducing the composition according to any one of (47) to (50) into a cell.

(54) A method for expressing the gp46 antigen or Tax antigen of human T-cell leukemia virus type 1 (HTLV-1) in vivo, comprising administering the composition according to any one of (47) to (51) to a mammal.

(55) A method for inducing an immune response to human T-cell leukemia virus type 1 (HTLV-1), comprising administering the composition according to (49) or (50) to a mammal.

(56) A method for preventing and/or treating infection with human T-cell leukemia virus type 1 (HTLV-1), comprising administering the composition according to any one of (49) to (52) to a mammal.

(57) A method for preventing the onset of and/or treating a disease caused by HTLV-1 selected from the group consisting of adult T cell leukemia/lymphoma (ATLL), HTLV-1 associated myelopathy (HAM) and HTLV-1 uveitis (HU), comprising administering the composition according to any one of (49) to (52) to a mammal.

(58) A peptide prepared by fusing the Tax antigen of human T-cell leukemia virus type 1 (HTLV-1) having a mutation, by which carcinogenicity deteriorates or disappears, with a signal peptide.

(59) The peptide according to (58), wherein the Tax antigen has at least one mutation selected from the group consisting of T130A, L131S, L319R and L320S relative to the amino acid sequence of SEQ ID NO: 11, and the amino acid sequence consisting of amino acids except the mutant amino acids has an identity of at least 95% with the amino acid sequence of SEQ ID NO: 11.

(60) The peptide according to (58) or (59), wherein the signal peptide is a peptide consisting of an amino acid sequence from positions 1 to 18 in the amino acid sequence of SEQ ID NO: 13.

(61) The particle according to any one of (31) to (37), wherein a nucleic acid capable of expressing the gp46 antigen or Tax antigen of human T-cell leukemia virus type 1 (HTLV-1) is mRNA comprising a structure consisting of a cap structure (Cap), a 5′-noncoding region (5′-UTR), a coding region of the gp46 antigen or Tax antigen and a 3′-noncoding region (3′-UTR).

(62) The particle according to (61), wherein the structure consisting of a cap structure (Cap), a 5′-noncoding region (5′-UTR), a coding region of the gp46 antigen or Tax antigen, and a 3′-noncoding region (3′-UTR), is a nucleotide sequence having an identity of at least 90% with a sequence from positions 1 to 1141 in SEQ ID NO: 17 or a sequence from positions 1 to 1222 in SEQ ID NO: 18.

(63) The particle according to (61), wherein the structure consisting of a cap structure (Cap), a 5′-noncoding region (5′-UTR), a coding region of the gp46 antigen or Tax antigen, and a 3′-noncoding region (3′-UTR) is a nucleotide sequence having an identity of at least 90% with a sequence from positions 1 to 1315 in SEQ ID NO: 20.

The specification incorporates the contents disclosed by JP Patent Application No. 2021-084942, based on which the priority of the present application claims.

### Advantageous Effects of Invention

Infection with human T-cell leukemia virus type 1 (HTLV-1) can be prevented and/or treated by the present invention. Also, a disease caused by infection with HTLV-1 can be prevented and/or treated by the present invention. The particle of the present invention has excellent properties in view of, e.g., metabolic stability, in-vitro activity, in-vivo activity, rapid exertion of a medicinal effect, persistency of a medicinal effect, physical stability, drug interaction and safety, and is useful as a medical drug for treating or preventing the diseases mentioned above.

### BRIEF DESCRIPTION OF DRAWINGS

FIG. 1 shows HIV-1 LTR transcriptional activity (A) and HTLV-1 LTR transcriptional activity (B) of a Tax wild type, a Tax mutant, and a secretory Tax mutant. “pHIV-1 LTR” represents an HIV-1 LTR reporter plasmid administration group and, “pHTLV-1 LTR” represents an HTLV-1 LTR reporter plasmid administration group. “Empty” represents a negative control group, pcDNA3.1+vector. The measurement is performed in triplicate. The vertical bars show average values of the measurement data and the error bars show SDs. The open circles on the bar graph show individual data obtained by triplicate measurement. “Tax WT”: Tax wild type, “Tax Mut”: a Tax mutant and “Sec-Tax Mut”: a secretory Tax mutant.

FIG. 2 shows the Tax-specific CTL induction level and anti-Tax antibody titer in the blood in C3H mice administered with the lipid particle of Example 12. FIG. 2A shows the Tax-specific CTL induction level and FIG. 2B shows the anti-Tax antibody titer in the blood.

FIG. 3 shows the anti-gp46 antibody titer in the blood and the anti-Tax antibody titer in the blood in monkeys. FIG. 3A shows the anti-gp46-specific antibody titer in the blood and FIG. 3B shows the anti-Tax antibody titer in the blood. A group consists of 4 monkeys. Data are shown at time points: 0W, 2W, 4W and 6W, which represent before administration, 2 weeks after the first administration, 4 weeks after the first administration (2 weeks after the second administration), 6 weeks after the first administration (2 weeks after the third administration), respectively. The bars show average values and open circles show individual data.

FIG. 4 shows gp46-specific and Tax-specific cell-mediated immune responses. A group consists of 4 monkeys. The vertical bars show average values of the measurement data and the error bars show SDs. The open circles on the bar graph show individual data.

FIG. 5 shows anti-HTLV-1 neutralization activity in monkey blood. “Assay negative control” shows the result of a sample not treated with an antibody and “Assay positive control” shows the result of a sample treated with an HTLV-1 neutralizing antibody commonly known in the technical field. A group consists of 4 monkeys. The numbers, #736, #741, #743 and #737 show individual monkey IDs in a negative control group. The numbers, #745, #742, #740 and #735 show individual monkey IDs in a group of monkeys administered with a preparation containing mRNA molecules of Example 5 and Example 6. The arrows indicate syncytia formation.

FIG. 6 shows the anti-gp46 antibody titer in the blood of C57BL/6 mice.

FIG. 7 shows gp46-specific and Tax-specific cell-mediated immune responses of C57BL/6 mice. FIG. 7A shows IFN-γ production levels and FIG. 7B shows IL-2 production levels.

FIG. 8 shows gp46 and Tax protein expression levels in CHO—S cells treated with the lipid particles of Example 9 to Example 12. FIG. 8A shows the expression levels in cell lysates and FIG. 8B shows expression levels in culture supernatants.

FIG. 9 shows the nucleotide sequence (SEQ ID NO: 1) of template plasmid DNA for IVT of HTLV-I SU gp46.

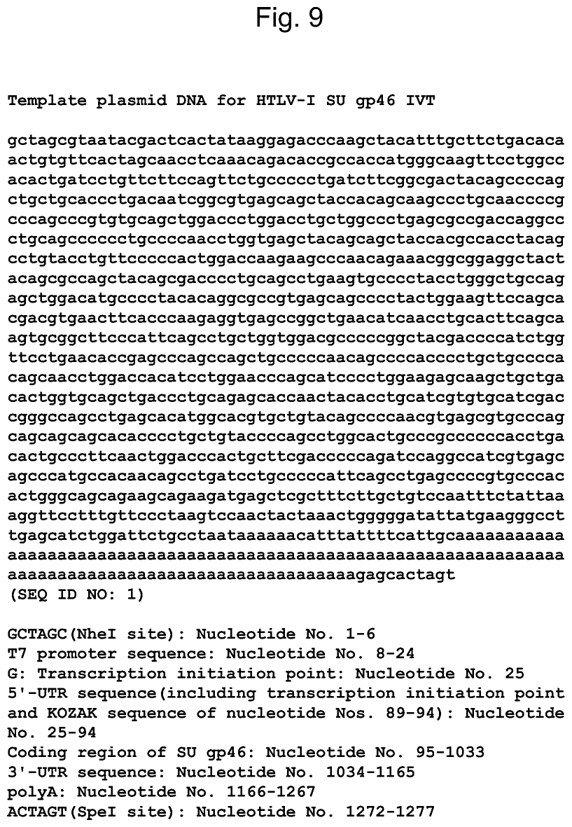

FIG. 10 shows the nucleotide sequence of a sense primer (SEQ ID NO: 2) and the nucleotide sequence of an antisense primer (SEQ ID NO: 3).

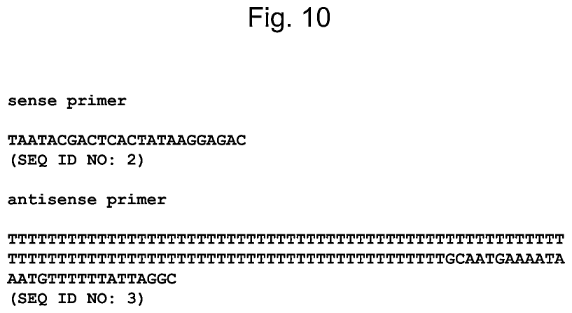

FIG. 11 shows the nucleotide sequence (SEQ ID NO: 4) of template DNA for in-vitro transcription (IVT) of SU gp46.

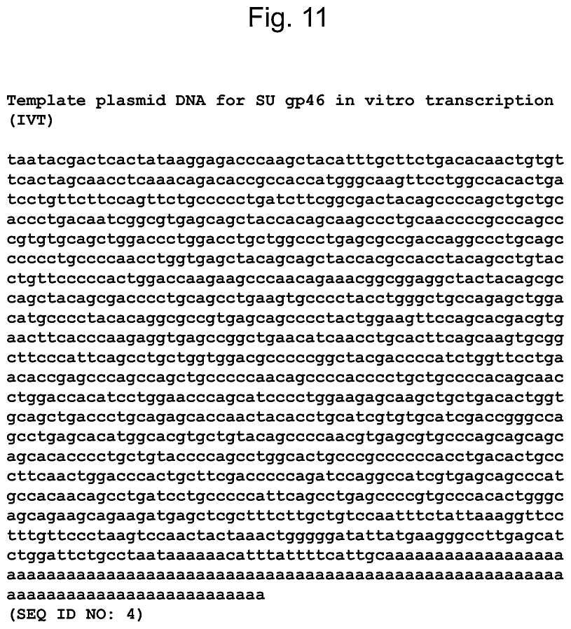

FIG. 12 shows the nucleotide sequence (SEQ ID NO: 5) of template plasmid DNA for IVT of HTLV-I gp46-Fib.

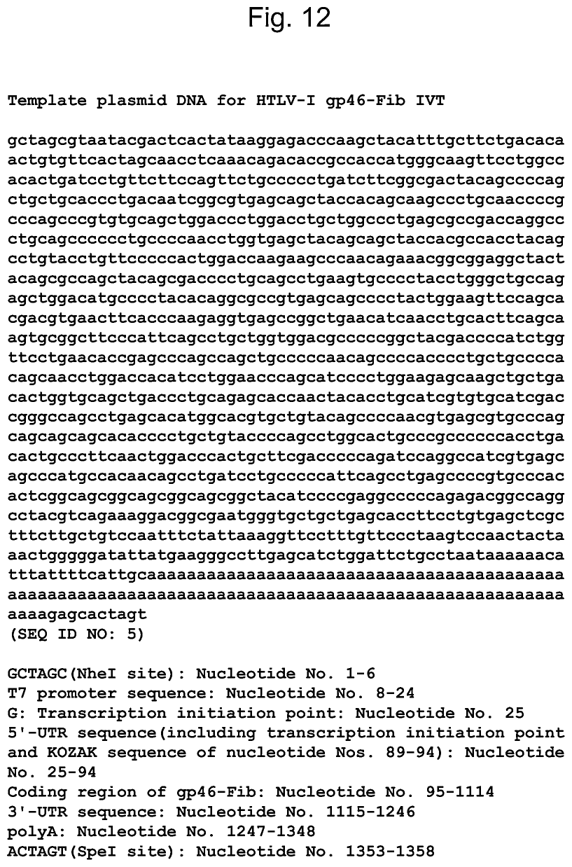

FIG. 13 shows the nucleotide sequence (SEQ ID NO: 6) of template DNA for in-vitro transcription (IVT) of gp46-Fib.

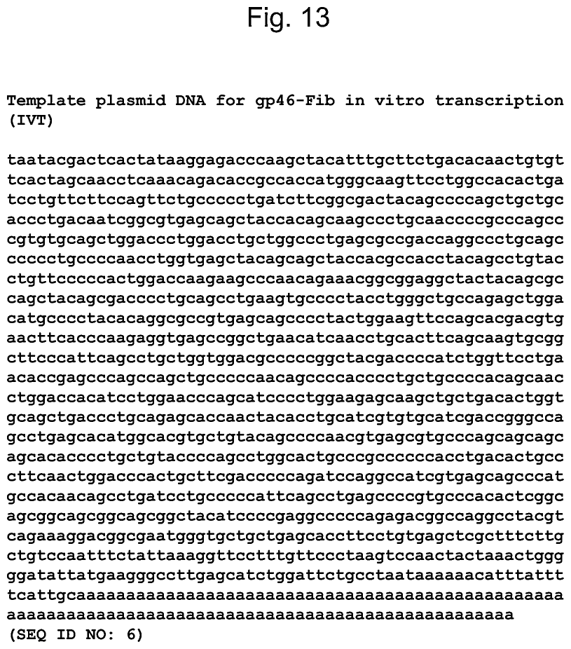

FIG. 14 shows the nucleotide sequence (SEQ ID NO: 7) of template plasmid DNA for IVT of HTLV-I dgp62-Fib.

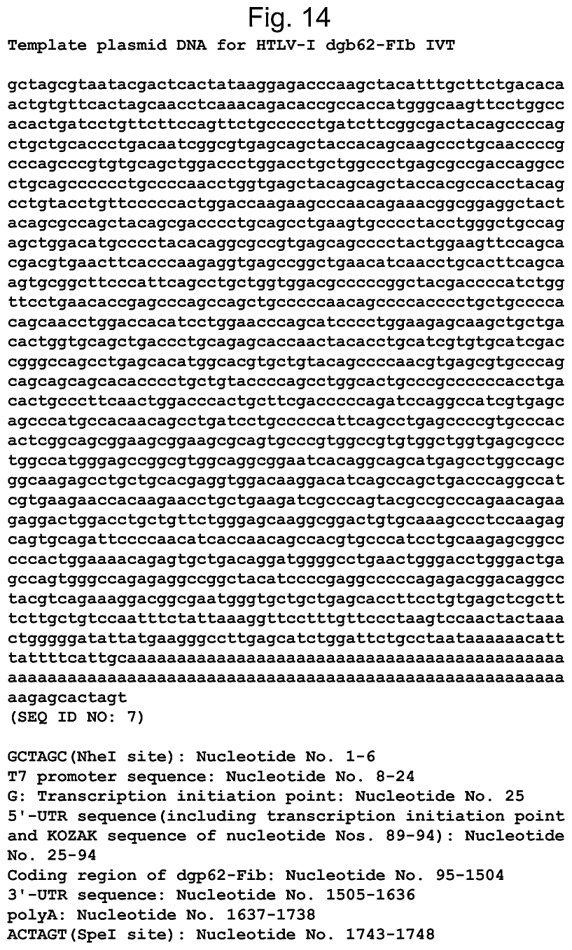

FIG. 15 shows the nucleotide sequence (SEQ ID NO: 8) of template DNA for in-vitro transcription (IVT) of dgp62-Fib.

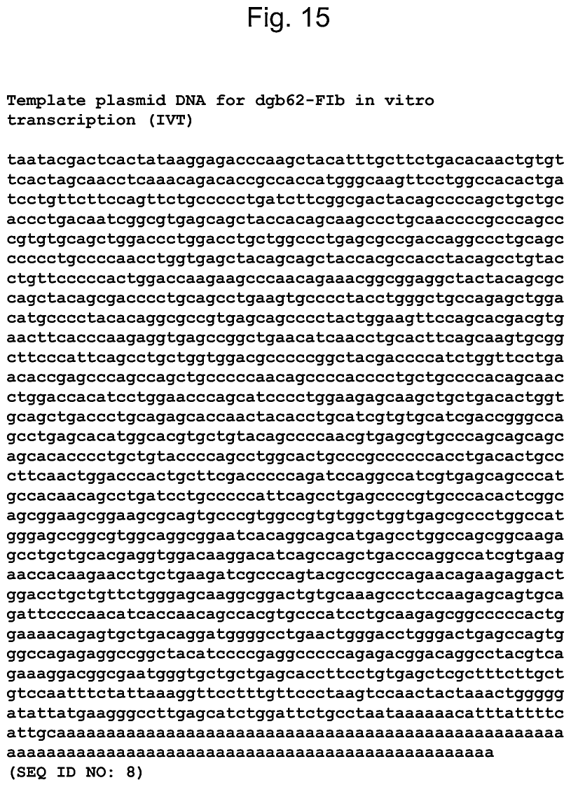

FIG. 16 shows the nucleotide sequence (SEQ ID NO: 9) of template plasmid DNA for IVT of an HTLV-I sec Tax mutant.

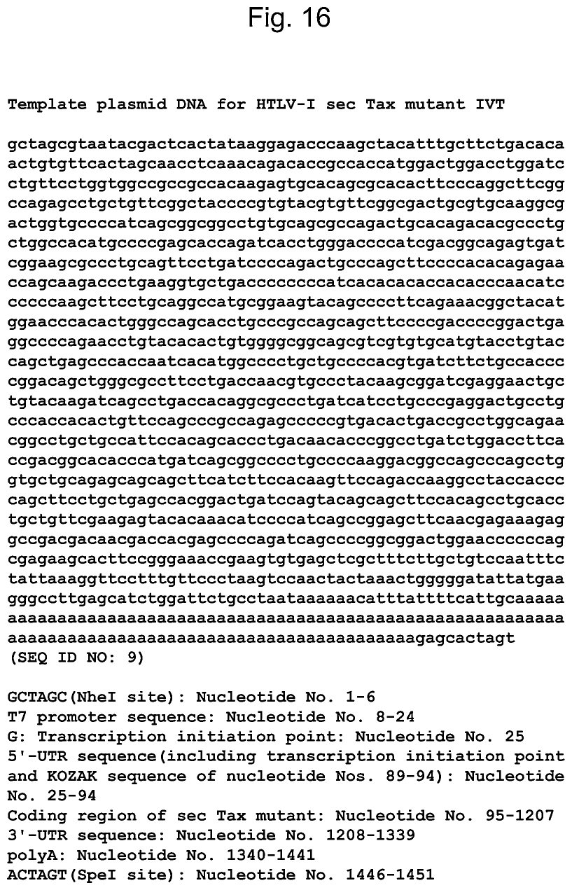

FIG. 17 shows the nucleotide sequence (SEQ ID NO: 10) of template DNA for in-vitro transcription (IVT) of a sec Tax mutant.

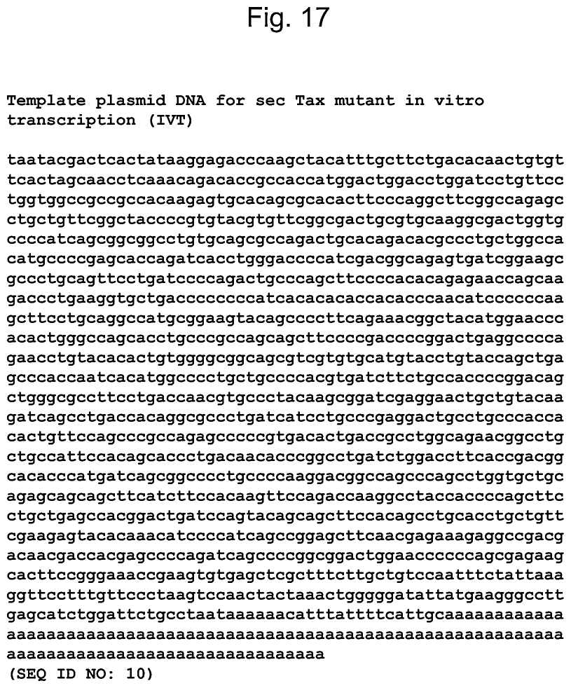

FIG. 18 shows the amino acid sequence (SEQ ID NO: 11) of a Tax wild type.

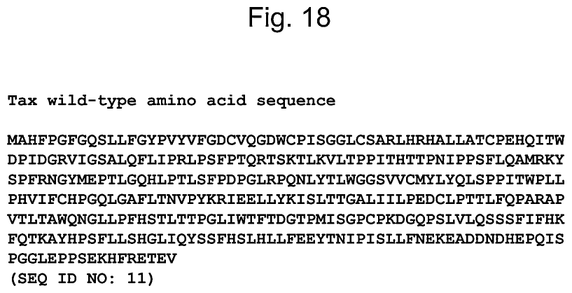

FIG. 19 shows the amino acid sequence (SEQ ID NO: 12) of a Tax mutant (T130A/L131S/L319R/L320S).

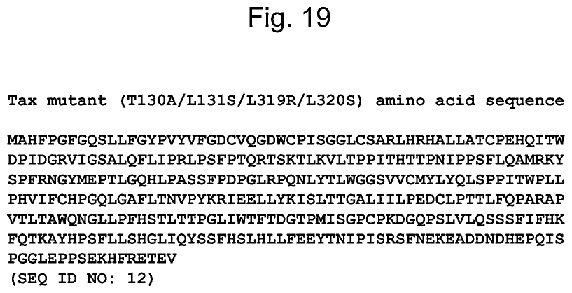

FIG. 20 shows the amino acid sequence (SEQ ID NO: 13) of a secretory Tax mutant (T130A/L131S/L319R/L320S).

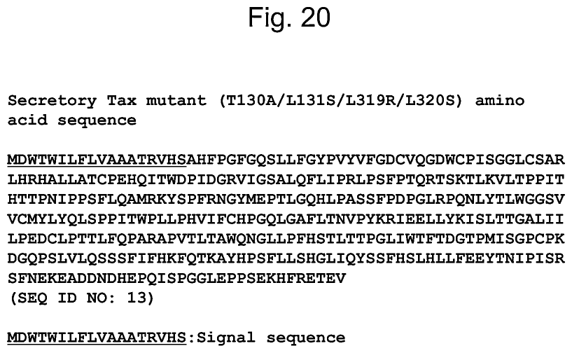

FIG. 21 shows the amino acid sequence (SEQ ID NO: 14) of gp46.

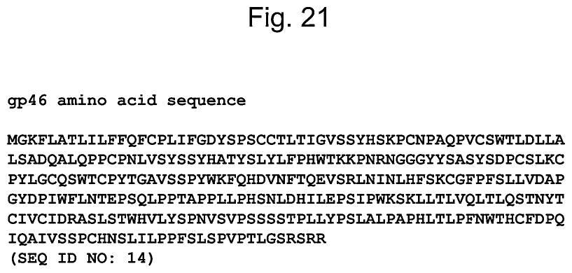

FIG. 22 shows the amino acid sequence (SEQ ID NO: 15) of gp46-Fib.

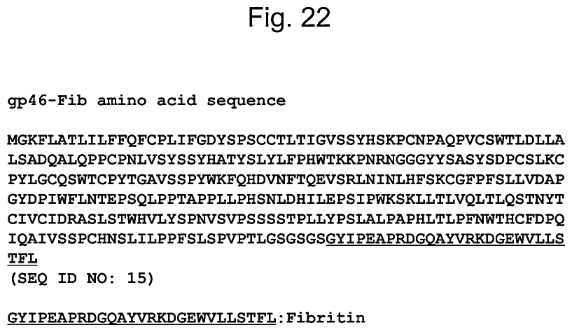

FIG. 23 shows the amino acid sequence (SEQ ID NO: 16) of dgp62-Fib.

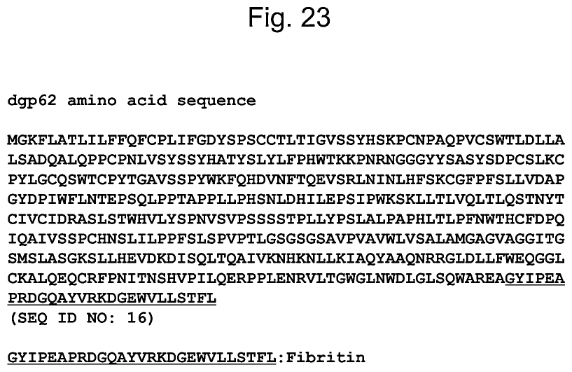

FIG. 24 shows the nucleotide sequence (SEQ ID NO: 17) of SU gp46 mRNA.

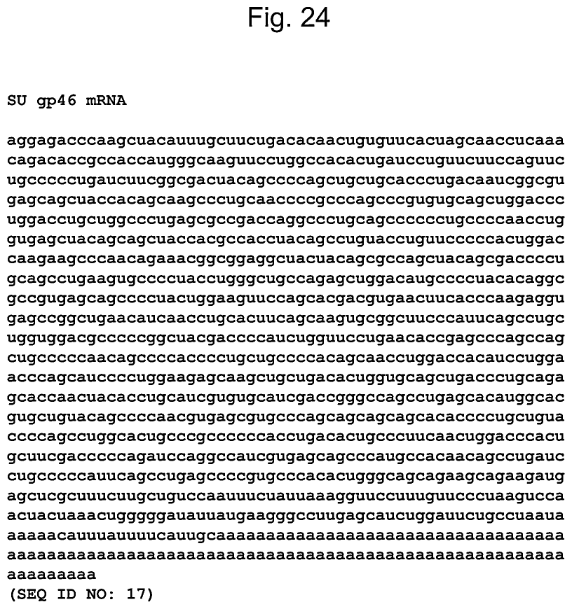

FIG. 25 shows the nucleotide sequence (SEQ ID NO: 18) of gp46-Fib mRNA.

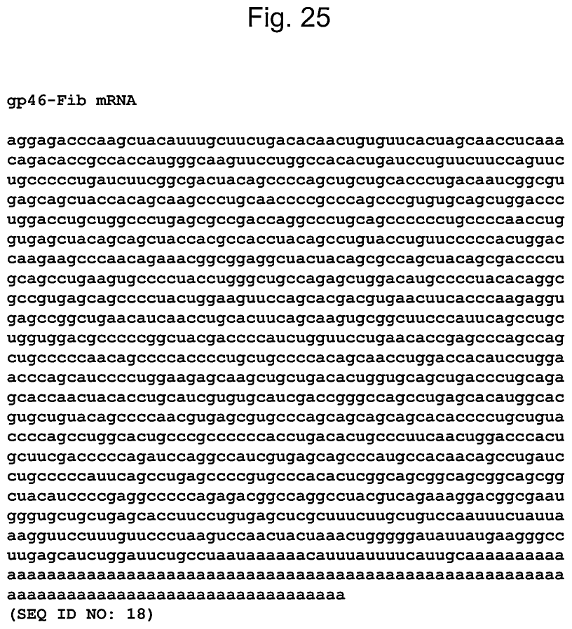

FIG. 26 shows the nucleotide sequence (SEQ ID NO: 19) of dgp62-Fib mRNA.

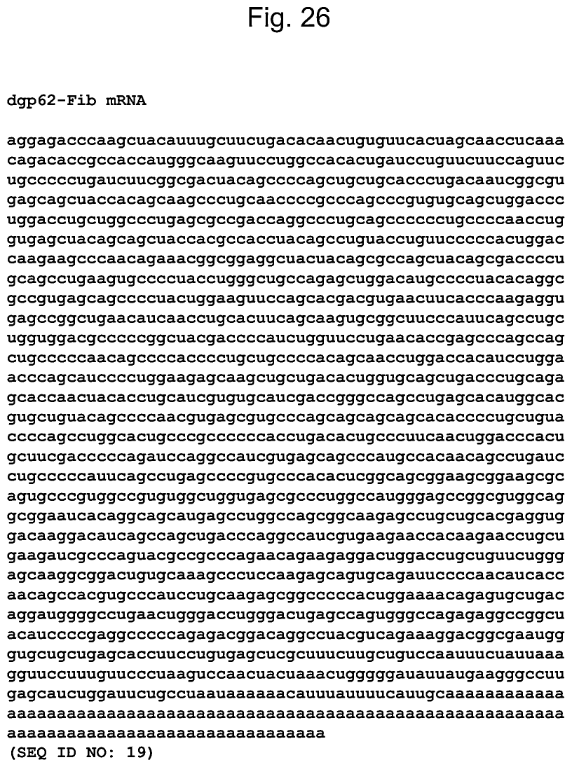

FIG. 27 shows the nucleotide sequence (SEQ ID NO: 20) of sec Tax mutant mRNA.

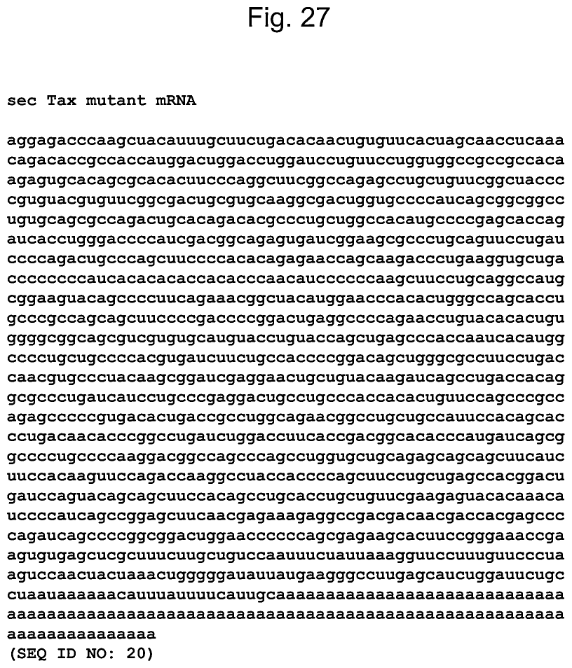

FIG. 28 shows the gp46 protein expression levels in CHO—S cells treated with Examples 30 to 34. The dotted line shows the OD value of Buffer as a negative control.

FIG. 29 shows the nucleotide sequence of the template DNA for gp46-Fib+polyA95 (SEQ ID NO: 21).

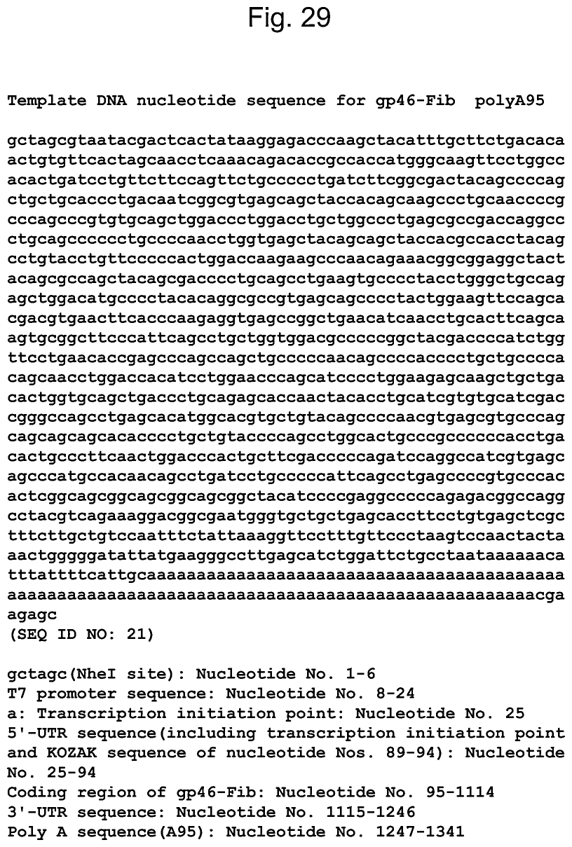

FIG. 30 shows the nucleotide sequence of the template DNA for gp46-Fib+polyA80 (SEQ ID NO: 22).

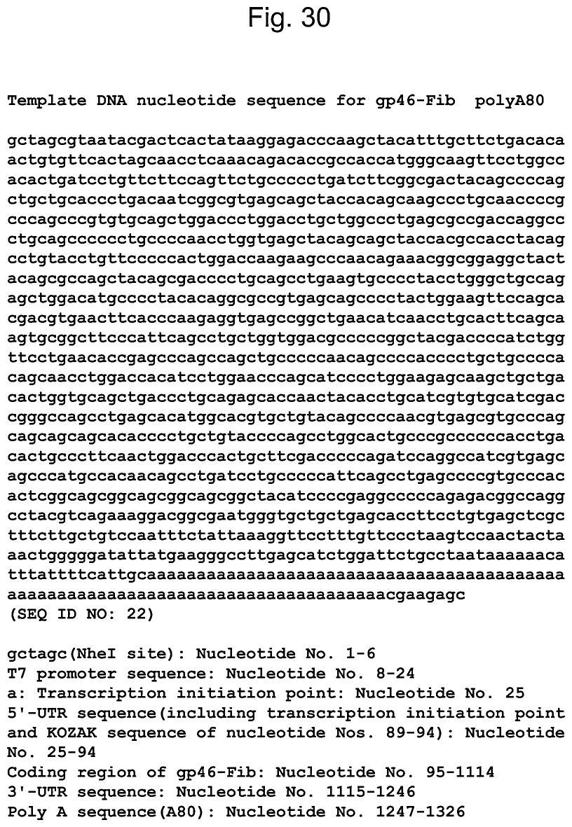

FIG. 31 shows the nucleotide sequence of the template DNA for gp46-Fib+polyA60 (SEQ ID NO: 23).

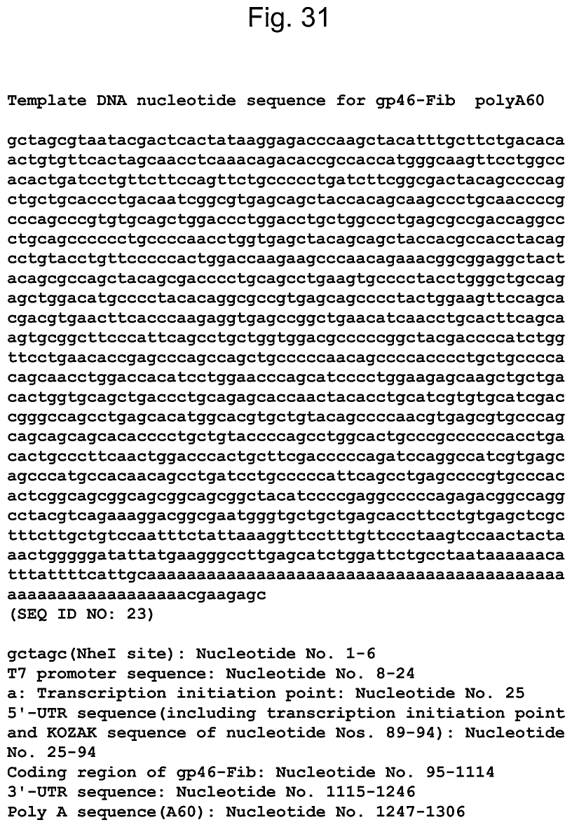

FIG. 32 shows the nucleotide sequence of the template DNA for gp46-Fib+polyA40 (SEQ ID NO: 24).

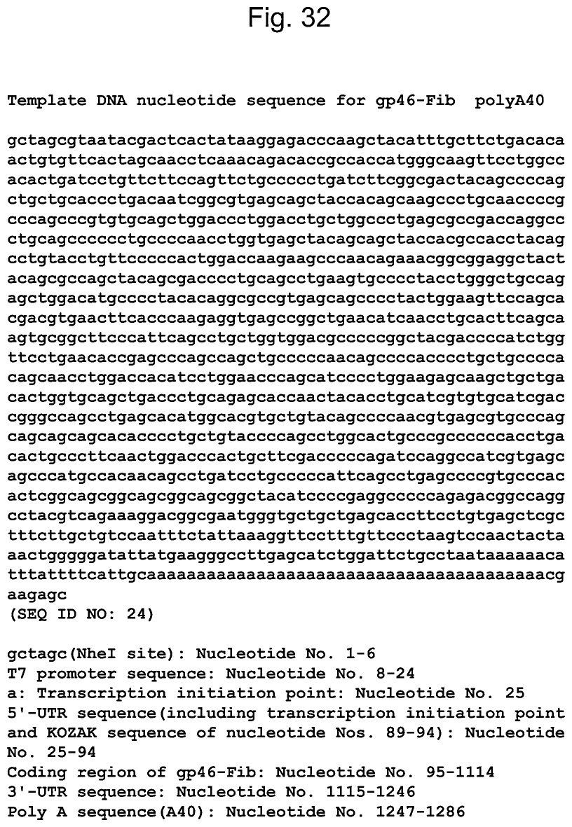

FIG. 33 shows the nucleotide sequence of the template DNA for gp46-Fib+polyA20 (SEQ ID NO: 25).

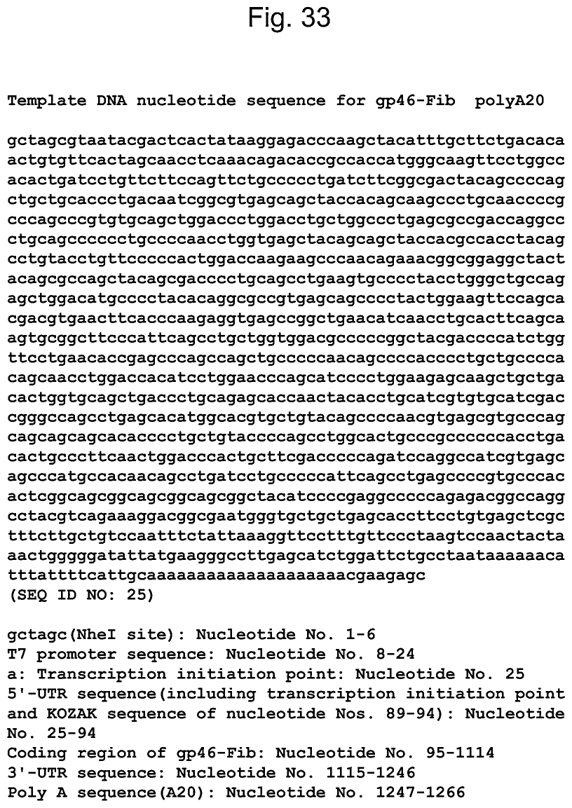

FIG. 34 shows the nucleotide sequence of the template DNA for sec Tax mutant+polyA95 (SEQ ID NO: 26).

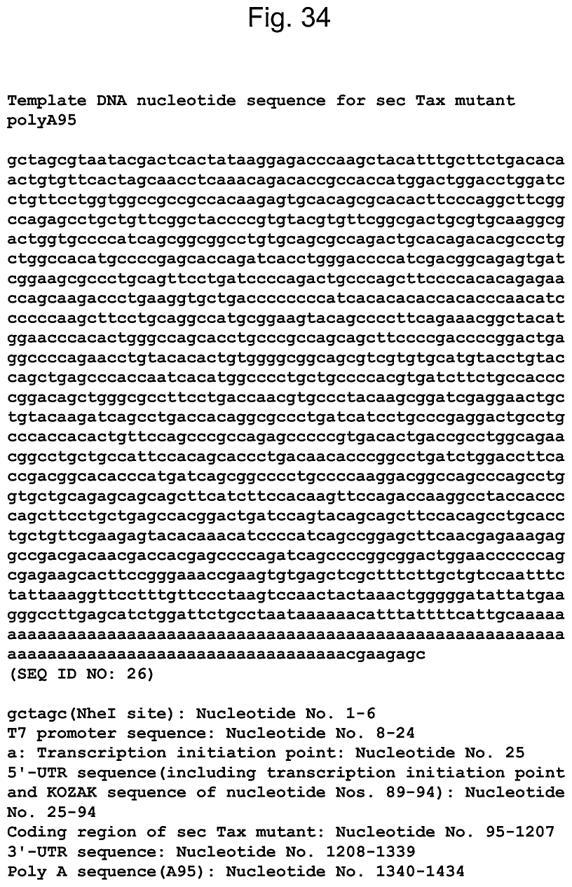

FIG. 35 shows the nucleotide sequence of the template DNA for sec Tax mutant+polyA80 (SEQ ID NO: 27).

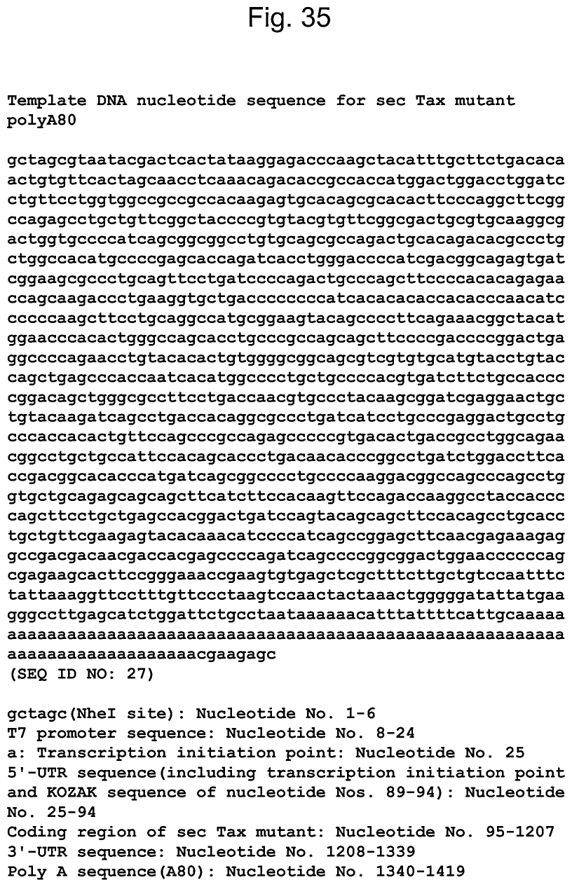

FIG. 36 shows the nucleotide sequence of the template DNA for sec Tax mutant+polyA60 (SEQ ID NO: 28).

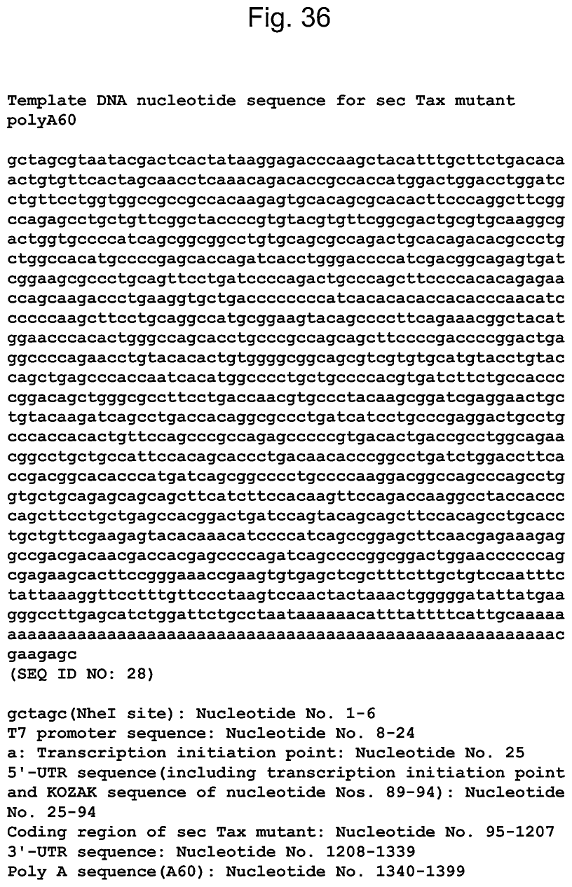

FIG. 37 shows the nucleotide sequence of the template DNA for sec Tax mutant+polyA40 (SEQ ID NO: 29).

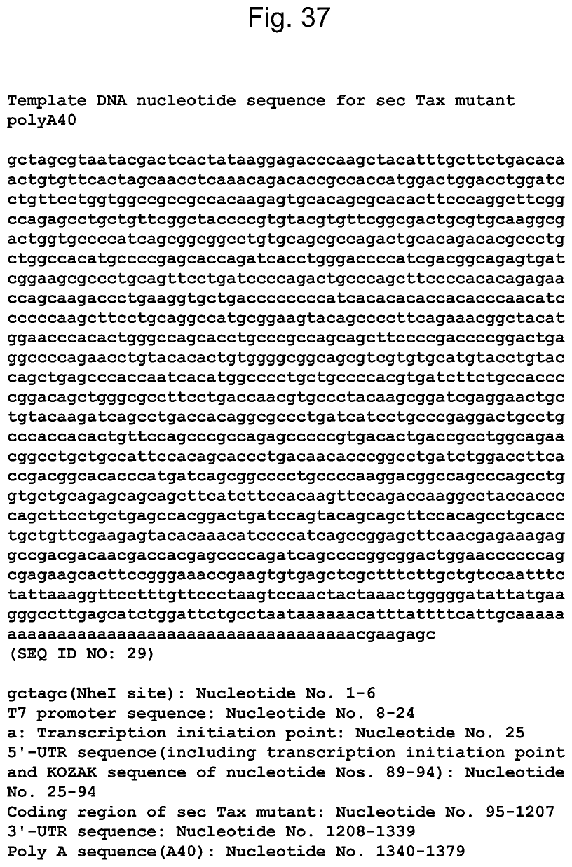

FIG. 38 shows the nucleotide sequence of the template DNA for sec Tax mutant+polyA20 (SEQ ID NO: 30).

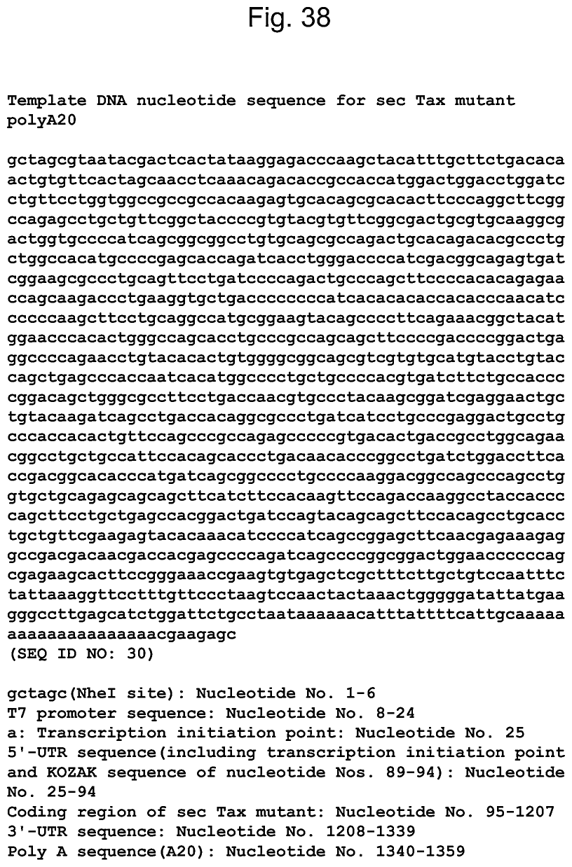

FIG. 39 shows the mRNA sequence of gp46-Fib+polyA95 (SEQ ID NO: 31).

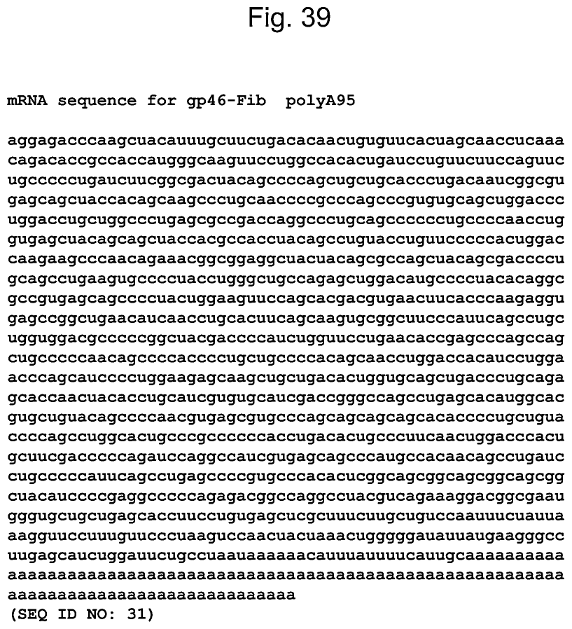

FIG. 40 shows the mRNA sequence of gp46-Fib+polyA80 (SEQ ID NO: 32).

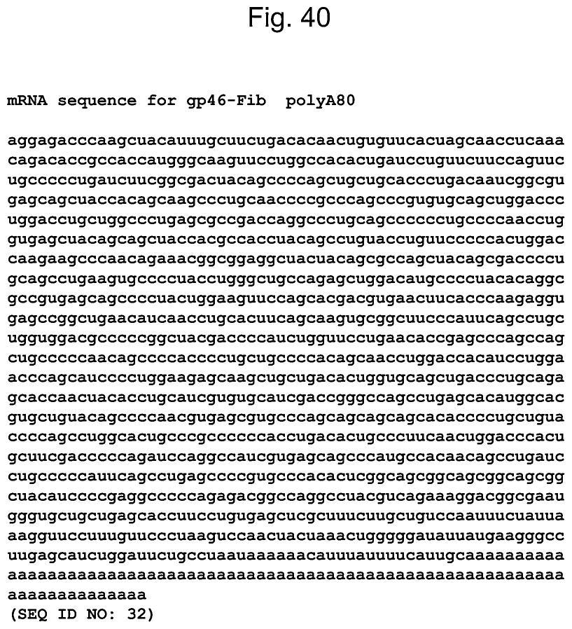

FIG. 41 shows the mRNA sequence of gp46-Fib+polyA60 (SEQ ID NO: 33).

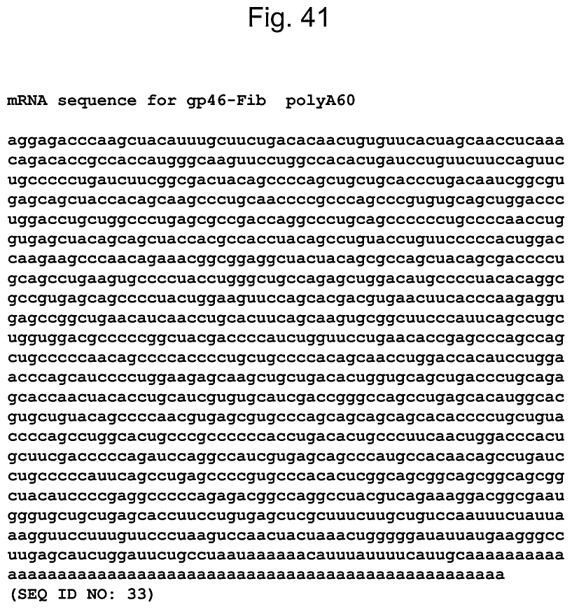

FIG. 42 shows the mRNA sequence of gp46-Fib+polyA40 (SEQ ID NO: 34).

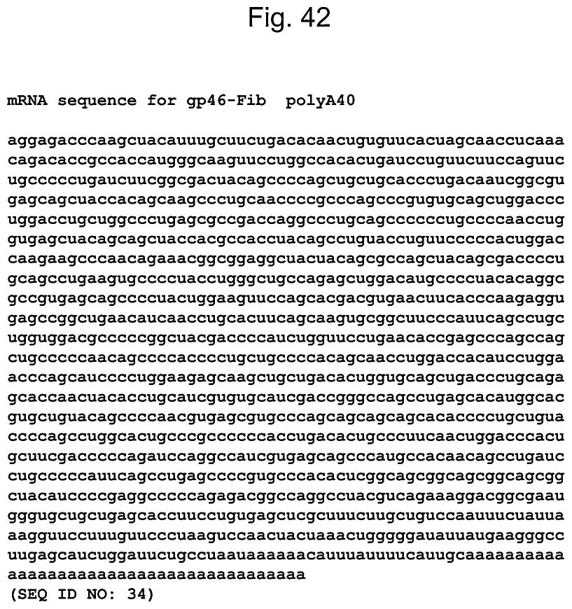

FIG. 43 shows the mRNA sequence of gp46-Fib+polyA20 (SEQ ID NO: 35).

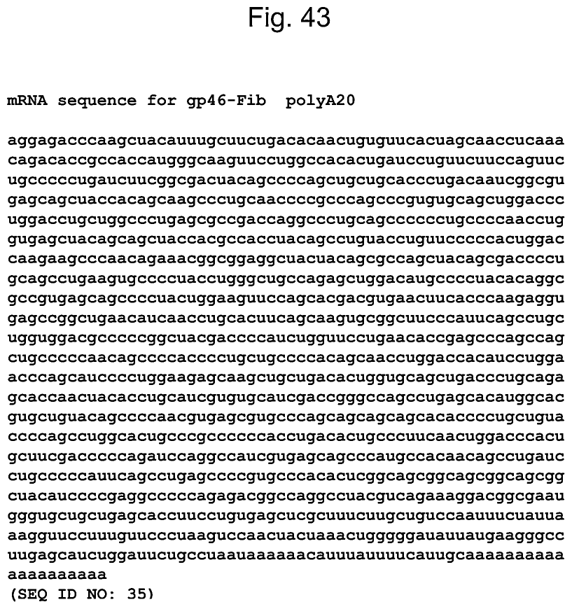

FIG. 44 shows the mRNA sequence of sec Tax mutant+polyA95 (SEQ ID NO: 36).

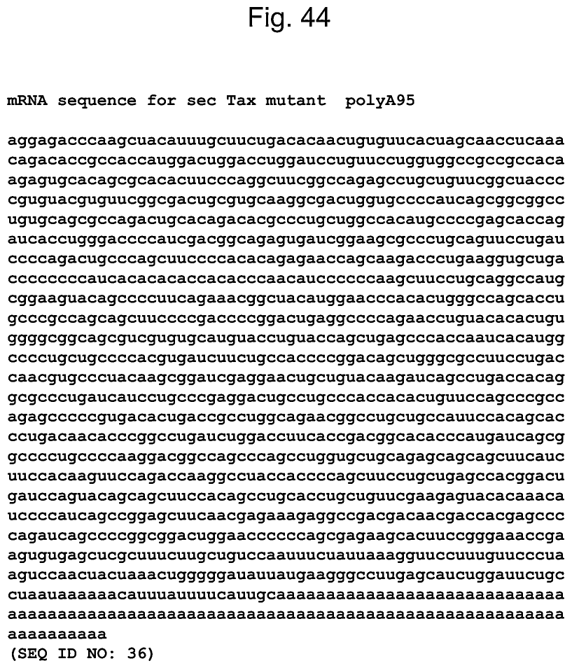

FIG. 45 shows the mRNA sequence of sec Tax mutant+polyA80 (SEQ ID NO: 37).

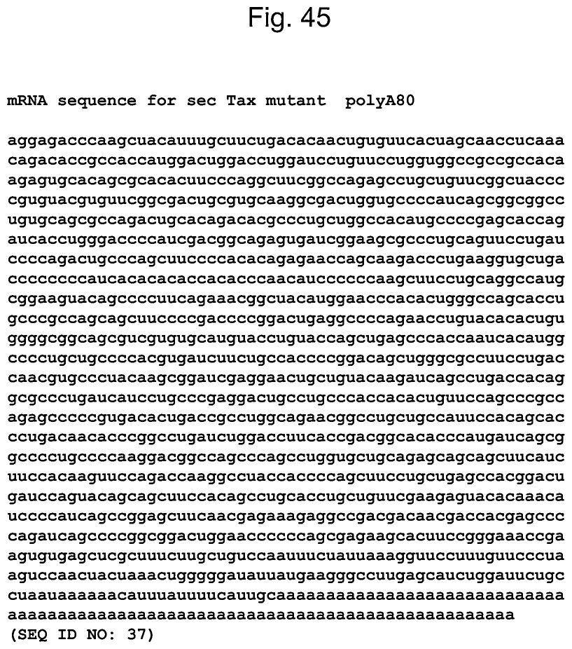

FIG. 46 shows the mRNA sequence of sec Tax mutant+polyA60 (SEQ ID NO: 38).

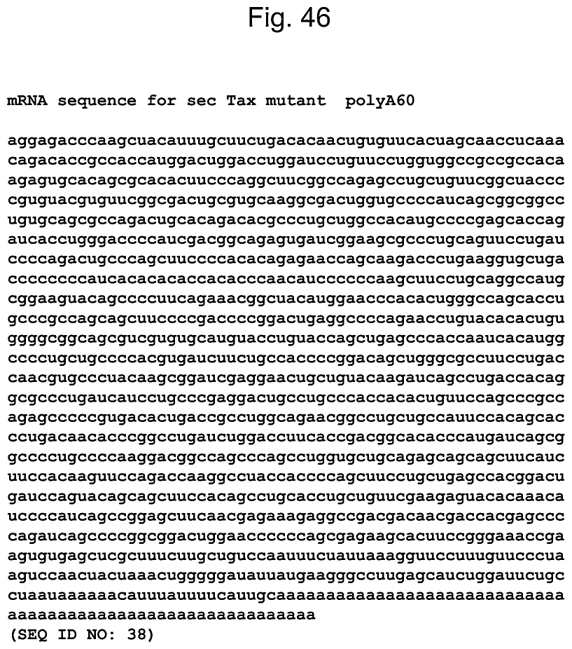

FIG. 47 shows the mRNA sequence of sec Tax mutant+polyA40 (SEQ ID NO: 39).

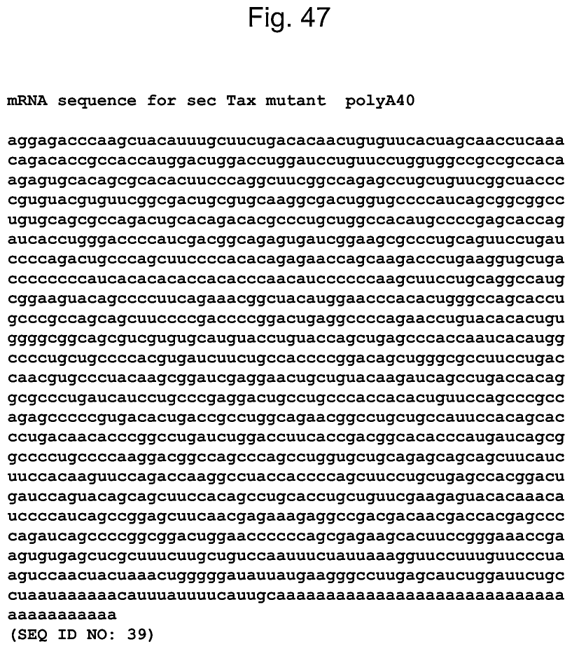

FIG. 48 shows the mRNA sequence of sec Tax mutant+polyA20 (SEQ ID NO: 40).

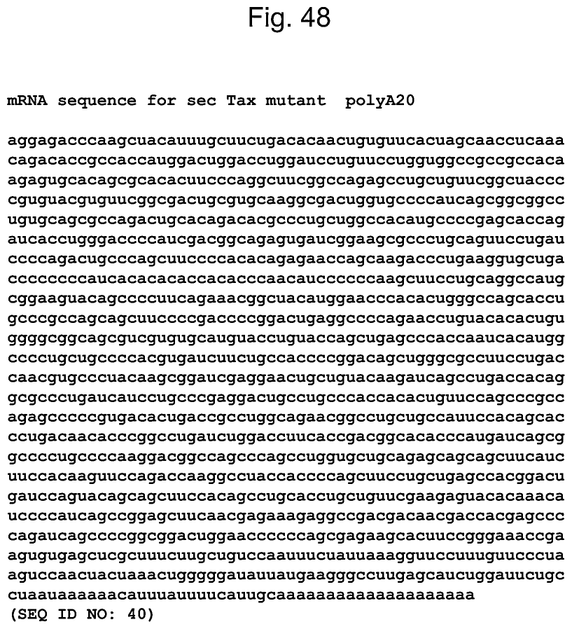

### DESCRIPTION OF EMBODIMENTS

Now, embodiments of the present invention will be more specifically described.

The present invention provides a lipid particle encapsulating a nucleic acid capable of expressing a gp46 antigen or a Tax antigen of HTLV-1, wherein the lipid comprises a cationic lipid represented by general formula (Ia):

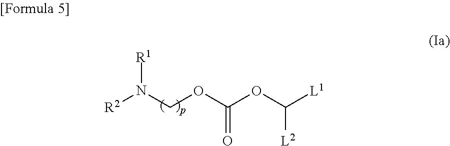

or a pharmaceutically acceptable salt thereof,
wherein
R¹ and R² each independently represent a C₁-C₃ alkyl group;
L¹ represents a C₁₇-C₁₉ alkenyl group optionally having one or more C₂-C₄ alkanoyloxy groups;
L² represents a C₁₀-C₁₉ alkyl group optionally having one or more C₂-C₄ alkanoyloxy groups, or a C₁₀-C₁₉ alkenyl group optionally having one or more C₂-C₄ alkanoyloxy groups; and
p is 3 or 4.

In general formula (Ia), R¹ and R² each independently represent a C₁-C₃ alkyl group, preferably both are methyl groups.

In general formula (Ia), p is 3 or 4, preferably 3.

In general formula (Ia), L¹ represents a C₁₇-C₁₉ alkenyl group optionally having one or more C₂-C₄ alkanoyloxy groups, and preferably, a C₁₇-C₁₉ alkenyl group optionally having one or more acetoxy groups. Examples of the group represented by L¹ include an (R)-11-acetyloxy-cis-8-heptadecenyl group, a cis-8-heptadecenyl group and an (8Z,11Z)-heptadecadienyl group.

In general formula (Ia), L² represents a C₁₀-C₁₉ alkyl group optionally having one or more C₂-C₄ alkanoyloxy groups, or a C₁₀-C₁₉ alkenyl group optionally having one or more C₂-C₄ alkanoyloxy groups, and preferably, a C₁₀-C₁₂ alkyl group optionally having one or more acetoxy groups, or a C₁₀-C₁₉ alkenyl group optionally having one or more acetoxy groups. Alternatively, in general formula (Ia), L² is preferably a C₁₀-C₁₂ alkyl group optionally having one or more acetoxy groups or a C₁₇-C₁₉ alkenyl group optionally having one or more acetoxy groups. Examples of the group represented by L² include a decyl group, a cis-7-decenyl group, a dodecyl group, and an (R)-11-acetyloxy-cis-8-heptadecenyl group.

Examples of the cationic lipid constituting the particle of the present invention include (7R,9Z,26Z,29R)-18-({[3-(dimethylamino)propoxy]carbonyl}oxy) pentatriaconta-9,26-diene-7,29-diyl diacetate, 3-dimethylaminopropyl(9Z,12Z)-octacosa-19,22-dien-11-yl carbonate, and (7R,9Z)-18-({[3-(dimethylamino)propyloxy]carbonyl}oxy)octacosa-9-en-7-yl acetate, which are respectively shown by the following structural formulas:

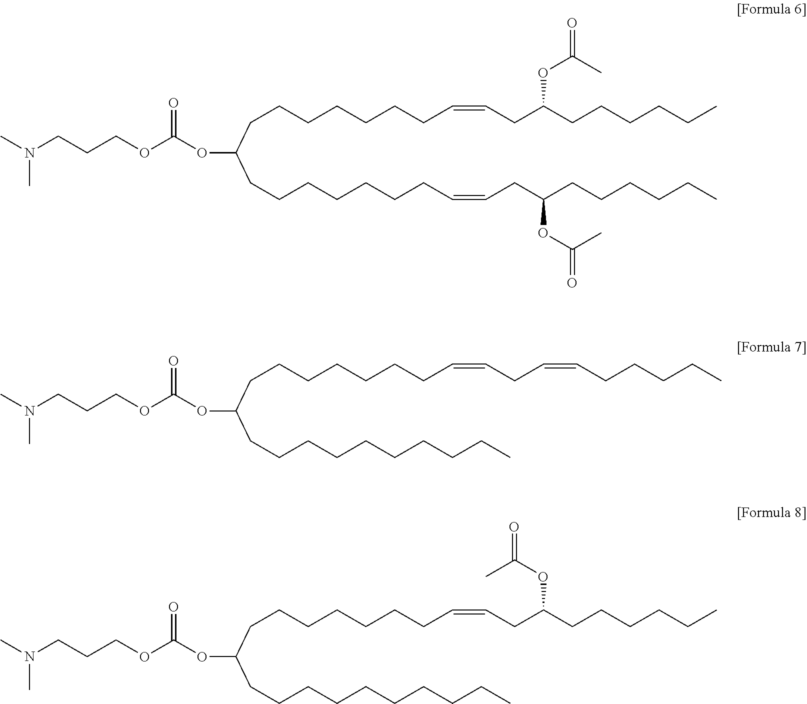

The cationic lipid represented by general formula (Ia) may be a single compound or a combination of two or more compounds.

A method for producing a cationic lipid represented by general formula (Ia) is disclosed in International Patent No. WO2015/005253.

The lipid of the present invention may further contain an amphipathic lipid, a sterol and a PEG lipid.

The amphipathic lipid is a lipid having an affinity for both polar and non-polar solvents. Examples of the amphipathic lipid include distearoylphosphatidylcholine, dioleoylphosphatidylcholine, dioleoylphosphatidylethanolamine and a combination of these. The amphipathic lipid to be used in the particle of the present invention is preferably distearoylphosphatidylcholine and/or dioleoylphosphatidylethanolamine, and more preferably, distearoylphosphatidylcholine.

The sterol refers to a sterol having a hydroxy group, such as cholesterol.

The PEG lipid is a lipid modified with PEG. Examples of the PEG lipid include 1,2-dimyristoyl-sn-glycerol methoxypolyethylene glycol and/or N-[methoxypoly(ethylene glycol)2000]carbamoyl]-1,2-dimyristyloxypropyl-3-amine and a combination of these. The PEG lipid is preferably 1,2-dimyristoyl-sn-glycerol methoxypolyethylene glycol. The average molecular weight of the PEG lipid, although it is not particularly limited, is, for example, 1000 to 5000, preferably 1500 to 3000, and more preferably, 1800 to 2200.

The lipid composition of an amphipathic lipid, a sterol, a cationic lipid and a PEG lipid, although it is not particularly limited, is as follows: for example, 5 to 25% of the amphipathic lipid; 10 to 55% of the sterol; 40 to 65% of the cationic lipid; and 1 to 5% of the PEG lipid, on a molar basis; preferably 10 to 25% of the amphipathic lipid; 10 to 55% of the sterol; 40 to 65% of the cationic lipid; and 1 to 5% of the PEG lipid on a molar basis; and more preferably 10 to 22.5% of the amphipathic lipid, 15 to 55% of the sterol; 40 to 65% of the cationic lipid; and 1 to 5% of the PEG lipid on a molar basis. In the above lipid composition, the ratio of the PEG lipid is more preferably 1 to 3%, further more preferably 1 to 2%, further more preferably 1.2 to 2%, further more preferably 1.25 to 2%, further more preferably 1.3 to 2%, and particularly preferably 1.5 to 2% on a molar basis. In the above lipid composition, the weight ratio of the total lipid to the nucleic acid, although it is not particularly limited, may be satisfactorily 15 to 30, preferably 15 to 25, more preferably 15 to 22.5, and further more preferably 17.5 to 22.5.

When 3-dimethylaminopropyl(9Z,12Z)-octacosa-19,22-dien-11-yl carbonate or (7R,9Z,26Z,29R)-18-({[3-(dimethylamino)propoxy]carbonyl}oxy) pentatriaconta-9,26-diene-7,29-diyl diacetate is used as the cationic lipid, the lipid composition of an amphipathic lipid, a sterol, a cationic lipid and a PEG lipid, although it is not particularly limited, contains, for example, 5 to 25% of the amphipathic lipid, 10 to 55% of the sterol, 40 to 65% of the cationic lipid, and 1 to 5% of the PEG lipid on a molar basis; preferably 15% or less of the amphipathic lipid, 20 to 55% of the sterol, 40 to 65% of the cationic lipid, and 1 to 5% of the PEG lipid; more preferably 5 to 15% of the amphipathic lipid, 35 to 50% of the sterol, 40 to 55% of the cationic lipid, and 1 to 3% of the PEG lipid, on a molar basis; further more preferably 10 to 15% of the amphipathic lipid, 35 to 45% of the sterol, 40 to 50% of the cationic lipid, and 1 to 2.5% of the PEG lipid, on a molar basis; and further more preferably 10 to 15% of the amphipathic lipid, 35 to 45% of the sterol, 40 to 50% of the cationic lipid, and 1 to 2% of the PEG lipid, on a molar basis. In the above lipid composition, the content of PEG lipid is further more preferably 1.2 to 2%, further more preferably 1.25 to 2%, further more preferably 1.3 to 2%, and further more preferably 1.5 to 2%. In the above lipid composition, the weight ratio of the total lipid to the nucleic acid, although it is not particularly limited, is satisfactorily 15 to 30, preferably 15 to 25, more preferably 15 to 22.5, and further more preferably 17.5 to 22.5.

When (7R,9Z)-18-({[3-(dimethylamino)propyloxy]carbonyl}oxy)octacosa-9-en-7-yl acetate is used as the cationic lipid, the lipid composition of an amphipathic lipid, a sterol, a cationic lipid and a PEG lipid, although it is not particularly limited, is, for example, 5 to 25% of the amphipathic lipid, 10 to 55% of the sterol, 40 to 65% of the cationic lipid, and 1 to 5% of the PEG lipid, on a molar basis; preferably 10 to 25% of the amphipathic lipid, 10 to 50% of the sterol, 40 to 65% of the cationic lipid, and 1 to 5% of the PEG lipid; more preferably 10 to 25% of the amphipathic lipid, 10 to 50% of the sterol, 40 to 65% of the cationic lipid, and 1 to 3% of the PEG lipid, on a molar basis; further more preferably 10 to 25% of the amphipathic lipid, 10 to 45% of the sterol, 42.5 to 65% of the cationic lipid, and 1 to 2.5% of the PEG lipid, on a molar basis; further more preferably 15 to 22.5% of the amphipathic lipid, 15 to 40% of the sterol, 45 to 65% of the cationic lipid, and 1 to 2% of the PEG lipid on a molar basis; and further more preferably 17.5 to 22.5% of the amphipathic lipid, 15 to 40% of the sterol, 45 to 65% of the cationic lipid, and 1 to 2% of the PEG lipid on a molar basis. In the above lipid composition, the content of PEG lipid is further more preferably 1.2 to 2%, further more preferably 1.25 to 2%, further more preferably 1.3 to 2%, and further more preferably 1.5 to 2%. In the above lipid composition, the weight ratio of the total lipid to the nucleic acid, although it is not particularly limited, is preferably 15 to 30, more preferably 15 to 25, further more preferably 15 to 22.5, and further more preferably 17.5 to 22.5.

The combination of the lipids in the present invention to be used may consist of an amphipathic lipid such as distearoylphosphatidylcholine, dioleoylphosphatidylcholine, or dioleoylphosphatidylethanolamine, a sterol such as cholesterol, a cationic lipid such as (7R,9Z,26Z,29R)-18-({[3-(dimethylamino)propoxy]carbonyl}oxy) pentatriaconta-9,26-diene-7,29-diyl diacetate, 3-dimethylaminopropyl(9Z,12Z)-octacosa-19,22-dien-11-yl carbonate, or (7R,9Z)-18-({[3-(dimethylamino)propyloxy]carbonyl}oxy)octacosa-9-en-7-yl acetate, and a PEG lipid such as 1,2-dimyristoyl-sn-glycerol methoxypolyethylene glycol or N-[methoxypoly(ethylene glycol)2000]carbamoyl]-1,2-dimyristyloxypropyl-3-amine. A preferable combination of the lipids to be used consists of an amphipathic lipid such as distearoylphosphatidylcholine or dioleoylphosphatidylethanolamine, a sterol such as cholesterol, a cationic lipid such as (7R,9Z,26Z,29R)-18-({[3-(dimethylamino)propoxy]carbonyl}oxy) pentatriaconta-9,26-diene-7,29-diyl diacetate, or (7R,9Z)-18-({[3-(dimethylamino)propyloxy]carbonyl}oxy)octacosa-9-en-7-yl acetate, and a PEG lipid such as 1,2-dimyristoyl-sn-glycerol methoxypolyethylene glycol. A more preferable combination of the lipids to be used in the present invention consists of an amphipathic lipid such as distearoylphosphatidylcholine, a sterol such as cholesterol, a cationic lipid such as (7R,9Z,26Z,29R)-18-({[3-(dimethylamino)propoxy]carbonyl}oxy) pentatriaconta-9,26-diene-7,29-diyl diacetate or (7R,9Z)-18-({[3-(dimethylamino)propyloxy]carbonyl}oxy)octacosa-9-en-7-yl acetate, and a PEG lipid such as 1,2-dimyristoyl-sn-glycerol methoxypolyethylene glycol.

In the present invention, the nucleic acid to be encapsulated in a lipid particle is a nucleic acid capable of expressing a gp46 antigen or Tax antigen of HTLV-1.

The amino acid sequence of the gp46 antigen of HTLV-1 is represented by SEQ ID NO: 14. The nucleic acid to be encapsulated in a lipid particle is preferably a nucleic acid encoding the gp46 antigen of HTLV-1, which consists of an amino acid sequence having an identity of at least 95%, preferably 96%, more preferably 97%, and more preferably 98% with the amino acid sequence of SEQ ID NO: 14.

The gp46 antigen may be a fusion protein with an oligomerization domain. The oligomerization domain is a protein responsible for multimerization of a protein fused thereto. Examples of the oligomerization domain include fibritin derived from T4 bacteriophage. Also, a trimeric domain of fibritin, a foldon domain, can be used. The amino acid sequence of a fusion protein of gp46 of HTLV-1 and fibritin is represented by SEQ ID NO: 15. The amino acid sequence from positions 313 to 339 in the amino acid sequence of SEQ ID NO: 15 is the amino acid sequence of fibritin. The oligomerization domain may be positioned at the C-terminal or N-terminal of the gp46 antigen.

The amino acid sequence of the wild-type Tax antigen of HTLV-1 is represented by SEQ ID NO: 11. The nucleic acid to be encapsulated in a lipid particle is preferably a nucleic acid encoding the Tax antigen of HTLV-1, which consists of an amino acid sequence having an identity of at least 95%, preferably 96%, and more preferably 97% with the amino acid sequence of SEQ ID NO: 11. Since the wild-type Tax antigen has HTLV-1 LTR transcriptional activity and carcinogenicity, toxicity, i.e., carcinogenicity, to a living body must be reduced or eliminated. For example, a Tax antigen having a mutation, by which carcinogenicity deteriorates or disappears, may be used. Since when Tax is secreted outside a cell, the carcinogenicity to the cells deteriorates, Tax may be a fusion protein with a signal peptide. Examples of the mutation, by which carcinogenicity deteriorates or disappears, include T130A, L131S, L319R and L320S. T130A and L131S are mutations, by which HIV-1 LTR and HTLV-1 transcriptional activities are damaged, whereas, L319R and L320S are mutations, by which HTLV-1 transcriptional activity is eliminated. The Tax antigen is satisfactory if it has at least one of these mutations, preferably 2, more preferably 3, and particularly preferably 4 mutations. The amino acid sequence of the mutant-type Tax antigen having the 4 mutations mentioned above is represented by SEQ ID NO: 12. As a signal peptide for secreting the Tax antigen outside a cell, an IgE secretory signal peptide is mentioned. The signal peptide is preferably fused to the N-terminal side of the Tax antigen. The amino acid sequence of a fusion protein of the signal peptide and the mutant-type Tax antigen having the 4 mutations is represented by SEQ ID NO: 13. The amino acid sequence from positions 1 to 18 in the amino acid sequence of SEQ ID NO: 13 is the amino acid sequence of an IgE secretory signal peptide.

The present invention includes a peptide of a secretory Tax mutant prepared by adding an IgE secretory signal to a Tax mutant, which is prepared by fusing a Tax antigen having at least one, preferably 2, more preferably 3, and particularly preferably 4 of the 4 amino acid mutations, to a signal peptide. The peptide is a peptide prepared by fusing the Tax antigen of human T-cell leukemia virus type 1 (HTLV-1) having a mutation, by which carcinogenicity deteriorates or disappears, and a signal peptide. Examples of the mutation, by which carcinogenicity deteriorates or disappears, include T130A, L131S, L319R and L320S. More specifically, the above peptide of the Tax antigen, is, for example, a peptide having at least one mutation selected from the group consisting of T130A, L131S, L319R and L320S relative to the amino acid sequence of SEQ ID NO: 11 and consisting of an amino acid sequence having an identity of at least 95%, preferably 96%, and more preferably 97% with the amino acid sequence of SEQ ID NO: 11 when the sequence consisting of amino acids except the mutant amino acids. The signal peptide for secreting the Tax antigen outside a cell is, e.g., an IgE secretory signal peptide. The signal peptide is fused preferably to the N-terminal side of the Tax antigen. The amino acid sequence from positions 1 to 18 in the amino acid sequence of SEQ ID NO: 13 is the amino acid sequence of an IgE secretory signal peptide. More specifically, the above peptide is, e.g., a peptide consisting of an amino acid sequence from positions 1 to 18 in the amino acid sequence of SEQ ID NO: 13.

In the present invention, the nucleic acid to be encapsulated in a lipid particle may be a nucleic acid capable of expressing a protein prepared by binding the gp46 antigen and the Tax antigen of HTLV-1 via a linker. The linker may be a linker consisting of an amino acid sequence containing a sequence cleavable with a protease, more specifically, containing an amino acid sequence that is recognized/cleaved by a protease, Furin. The sequence cleavable with a protease is not limited as long as it can be cleaved by a Furin protein. Examples of the sequence cleavable with a protease include a sequence represented by R-X-K/R-R (R represents arginine, K lysine, X arbitrary amino acid) (J. Biol. Chem. 1992, 267, 16396; J. Biol. Chem. 1991, 266, 12127).

The identity of an amino acid sequence refers to a numerical value representing a matching rate of amino acids to the full-length sequence, provided that sequences in which the corresponding amino acids completely match are defined as the same amino acid sequences. The sequence identity in the present invention is calculated using sequence analysis software, GENETYX-SV/RC (made by Genetics Co., Ltd.). The algorithm employed in the software is commonly used in the technical field. The amino acid sequence encoded by the nucleic acid to be encapsulated in a lipid particle of the present invention may have a mutation (substitution), deletion, insertion and/or addition of an amino acid, as long as the amino acid sequence has an identity above a certain percentage with those of SEQ ID NOs: 11 to 16.

The amino acid sequence encoded by the nucleic acid to be encapsulated in a lipid particle of the present invention and having the aforementioned sequence identity, may have a substitution, deletion, insertion and/or addition at several sites (preferably 5 sites or less, more preferably 3, 2 or 1 site) at a ratio of several amino acids per site (preferably 10 or less, more preferably 7 or less, further preferably 5, 4, 3, 2 or 1 amino acid), in each of the amino acid sequences of SEQ ID NOs: 11 to 16.

The nucleic acid capable of expressing the gp46 antigen or Tax antigen of HTLV-1 is preferably mRNA containing a cap structure (Cap), a 5′-noncoding region (5′-UTR), a gp46 or Tax coding region, a 3′-noncoding region (3′-UTR) and a poly A tail (polyA). The KOZAK sequence may be present 5′ to the gp46 or Tax coding region. The cap structure (Cap) is a site present on the 5′-end of mRNA molecules in many eukaryotes and having a 7-methyl guanosine structure. Examples of the cap structure include Cap-0, Cap-1, Cap-2 and a cap structure using ARCA (Anti-Reverse Cap Analog). The cap structures are shown in the following formulas.

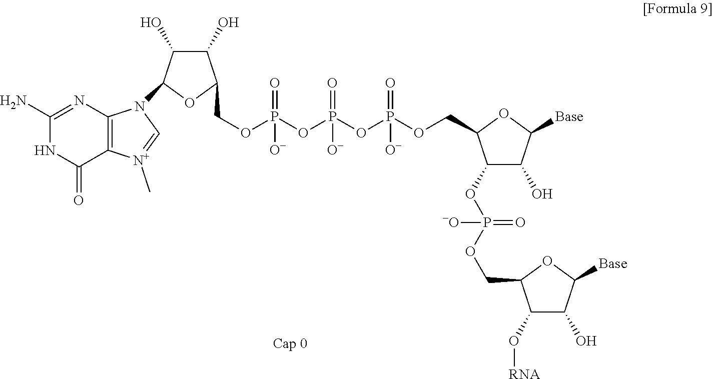

wherein “Base” represents an unmodified or modified nucleobase, and RNA represents a polynucleotide.

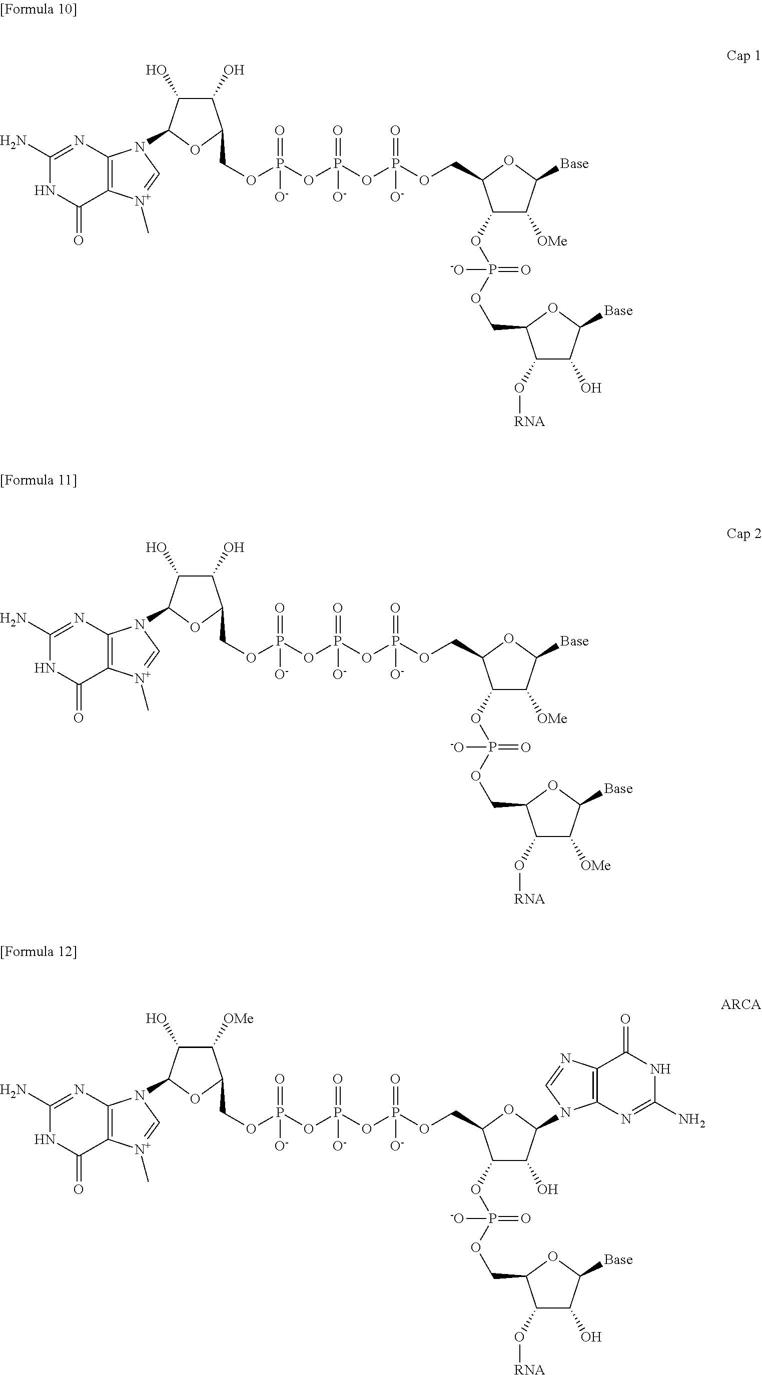

The cap structure of mRNA of the present invention is preferably Cap-0 or Cap-1, and more preferably, Cap-1. As the sequence of the 5′-noncoding region (5′-UTR), for example, a sequence containing the 5′-noncoding region of the human β globin gene can be used. For example, the sequence of the 5′-noncoding region of the human β globin gene is a sequence of nucleotide Nos. 15 to 64 in the sequence of SEQ ID NO: 17. The sequence of the coding region for gp46 is a sequence capable of expressing all or part of the amino acid sequence of the gp46 antigen; may contain an initiation codon and/or a termination codon; and is, for example, the sequence of nucleotide Nos. 71 to 1009 in the sequence of SEQ ID NO: 17. The sequence of the coding region for gp46 may be a sequence to which the sequence of fibritin is ligated, for example, the sequence of nucleotide Nos. 71 to 1090 in SEQ ID NO: 18. The sequence of the coding region for dgp62 is a sequence capable of expressing all or part of the amino acid sequence of the dgp62 antigen; may contain an initiation codon and/or a termination codon; and is, for example, the sequence of nucleotide Nos. 71 to 1396 in the sequence of SEQ ID NO: 19. The sequence of the coding region for dgp62 may be a sequence to which the sequence of fibritin is ligated, for example, represented by the sequence of nucleotide Nos. 71 to 1480 in the sequence of SEQ ID NO: 19. The sequence of the coding region for Tax is a sequence capable of expressing all or part of the amino acid sequence of the Tax antigen; and may contain an initiation codon and/or a termination codon. The Tax antigen contains 4 mutations such as T130A, L131S, L319R and L320S4. A sequence obtained by ligating the sequence of the coding region of mutant-type Tax antigen having the 4 mutations and a sequence encoding an IgE secretory signal peptide for secreting the Tax antigen outside a cell is mentioned, that is, the sequence of nucleotide Nos. 71 to 1183 in the sequence of SEQ ID NO: 20. As the sequence of the 3′-noncoding region (3′-UTR), for example, the 3′-noncoding region of the human β globin gene can be used. The sequence is, for example, the sequence of nucleotide Nos. 1010 to 1141 in the sequence of SEQ ID NO: 17. The sequence of poly A tail (polyA) is, for example, a sequence of nucleotide Nos. 1142 to 1241 in the sequence of SEQ ID NO: 17. The sequences of the cap structure (Cap), 5′-noncoding region (5′-UTR), gp46 or Tax coding region, 3′-noncoding region (3′-UTR) and poly A tail (polyA) may be modified. The sequence of the nucleic acid capable of expressing the gp46 antigen satisfactorily consists of a nucleotide sequence having an identity of at least 90%, preferably 95% and more preferably 97% with the sequence of SEQ ID NO: 17. The sequence of the nucleic acid capable of expressing the Tax antigen satisfactorily consists of a nucleotide sequence having an identity of at least 90%, preferably 95% and more preferably 97% with the sequence of SEQ ID NO: 20.

The length of the poly A tail, although it is not limited, is for example, a length of 10 to 250 nucleotides, preferably 15 to 120 nucleotides, further preferably 15 to 115 nucleotides, and particularly preferably 20 to 110 nucleotides.

The mRNA of the present invention has a sequence containing the cap structure (Cap), 5′-noncoding region (5′-UTR), gp46 or Tax coding region and 3′-noncoding region (3′-UTR). The portion consisting of the cap structure (Cap), 5′-noncoding region (5′-UTR), gp46 antigen or Tax antigen coding region and 3′-noncoding region (3′-UTR) may be mRNA consisting of a nucleotide sequence having an identity of at least 90%, preferably 95%, and more preferably 97% with the sequence from positions 1 to 1141 in SEQ ID NO: 17, the sequence from positions 1 to 1222 in SEQ ID NO: 18 or the sequence from positions 1 to 1315 in SEQ ID NO: 20.

The nucleic acid to be encapsulated in a lipid particle may be present in any form as long as it is a nucleic acid expressing the gp46 antigen or Tax antigen of HTLV-1. Examples of the form of the nucleic acid include a single-stranded DNA, single-stranded RNA (for example, mRNA), a single-stranded polynucleotide consisting of a combination of DNA and RNA, double-stranded DNA, double-stranded RNA, a hybrid polynucleotide consisting of DNA-RNA, and a double-stranded polynucleotide consisting of two types of polynucleotides, i.e., a combination of DNA and RNA. The nucleic acid is preferably mRNA.

The nucleotides constituting the nucleic acid to be encapsulated in a lipid particle may be natural-type nucleotides or modified nucleotides. At least one of the modified nucleotides is preferably contained.

The modified nucleotide is satisfactory as long as any one of a nucleotide, a sugar and a phosphodiester bond is modified. The number of modification sites may be 1 or 2 or more.

Examples of the nucleotide modification include 5-methylation of cytosine, 5-fluorination, N4-methylation, 5-methylation (thymine) of uracil, 5-fluorination, N6-methylation of adenine and N2-methylation of guanine.

Examples of the sugar modification include 2′-O-methylation of D-ribofuranose.

Examples of the modified phosphodiester-bond include a phosphorothioate bond.

The modified nucleotide is preferably a nucleotide having a modified nucleobase. For example, a pyrimidine nucleotide substituted at position 5, and pseudouridine optionally substituted at position 1 are satisfactory. Examples of the modified nucleotide may be 5-methylcytidine, 5-methoxyuridine, 5-methyluridine, pseudouridine, and a 1-alkylpseudouridine. The 1-alkylpseudouridine may be 1-(C1-C6 alkyl) pseudouridine, and preferably, 1-methylpseudouridine or 1-ethyl pseudouridine. As more preferable modified nucleotide, 5-methylcytidine, 5-methyluridine, and 1-methylpseudouridine are mentioned. As particularly preferable modified nucleotide, a combination of 5-methylcytidine and 5-methyluridine, or a combination of 5-methylcytidine and 1-methylpseudouridine is mentioned.

The nucleic acid capable of expressing the gp46 antigen or Tax antigen of HTLV-1 of the present invention can be produced from DNA having a desired nucleotide sequence by an in-vitro transcription reaction. The reagents required for the in-vitro transcription such as enzyme, buffer, and, a nucleoside-5′-triphosphate mixture (adenosine-5′-triphosphate (ATP), guanosine-5′-triphosphate (GTP), cytidine-5′-triphosphate (CTP) and uridine-5′-triphosphate (UTP)) are commercially available (e.g., AmpliScribeT7 High Yield Transcription Kit (Epicentre), mMESSAGE mMACHINE T7 Ultra Kit (Life technologies). The DNA for use in producing a single-stranded RNA may be cloned DNA such as plasmid DNA or a DNA fragment. The plasmid DNA or DNA fragment may be a commercially available one or can be produced by a method commonly known in the technical field (for example, method disclosed in Sambrook, J. et al., Molecular Cloning a Laboratory Manual second edition (1989), Rashtchian, A., Current Opinion in Biotechnology, 1995, 6 (1), 30-36, Gibson D. G. et al., Science, 2008, 319 (5867), 1215-1220).

In order to obtain mRNA improved in stability and/or safety, part or whole unmodified nucleotides in mRNA may be replaced with modified nucleotides, by substituting part or whole unmodified nucleoside-5′-triphosphate with modified nucleoside-5′-triphosphate in in-vitro transcription reaction (Kormann, M., Nature Biotechnology, 2011, 29, 154-157).

In order to obtain mRNA improved in stability and/or safety, a cap structure (Cap-0 structure mentioned above) can be introduced into the 5′-end of mRNA by a method using a capping enzyme after the in-vitro transcription reaction. Cap-0 may be converted into Cap-1 by a method of allowing 2′-O-methyltransferase to act on mRNA having Cap-0. As the capping enzyme and 2′-O-methyltransferase, commercially available ones (for example, Vaccinia Capping System, M2080; mRNA Cap 2′-O-Methyltransferase, M0366, both manufactured by New England Biolab) may be used. When a commercially available product is used, mRNA having a cap structure can be produced in accordance with the protocol attached to the product.

The cap structure at the 5′-end of mRNA can be introduced also by a method different from the enzymatic method. For example, a cap analogous structure that ARCA has or a Cap-1 structure derived from CleanCap can be introduced into mRNA by adding ARCA or CleanCap (registered trademark) in an in-vitro transcription reaction. As the ARCA and CleanCap, commercially available products can be used (ARCA, N-7003; CleanCap Reagent AG, N-7113, both are manufactured by TriLink BioTechnologies). When a commercially available product is used, mRNA having a cap structure can be manufactured in accordance with the protocol attached to the product.

In the present invention, the nucleic acid to be encapsulated in a lipid particle may be purified with a method such as desalting, HPLC (reverse phase, gel filtration, ion exchange, affinity), PAGE and ultrafiltration. Since impurities are removed by a purification treatment, production of inflammatory cytokines can be decreased in a living body administered with the nucleic acid.

The nucleic acid-encapsulated lipid particle of the present invention can be produced by a method such as a thin-film method, a reverse phase evaporation method, an ethanol injection method, an ether injection method, a dehydration-rehydration method, a surfactant dialysis method, a hydration method and a freeze-thaw method. For example, the nucleic acid-encapsulated lipid particle can be produced in accordance with a method disclosed in International Publication No. WO2015/005253. The nucleic acid-encapsulated lipid particle of the present invention can be produced by mixing a nucleic acid solution and a lipid solution in a micro-channel such as NanoAssemblr (registered trademark) of Precision Nanosystems, in accordance with the method set forth in the protocol attached thereto.

The average particle size of the particles of the present invention is satisfactory 30 nm to 300 nm, preferably 30 to 200 nm, and more preferably 30 to 100 nm. The average particle size can be obtained by determining a volume average particle size by a device such as Zeta Potential/Particle Sizer NICOMP (registered trademark) 380ZLS (PARTICLE SIZING SYSTEMS) and based on the principle of dynamic light scattering.

The particle of the present invention can be used for producing a composition for preventing and/or treating a disease caused by HTLV-1 infection. Examples of the disease caused by HTLV-1 infection include adult T cell leukemia (ATL), HTLV-1-associated myelopathy (HAM) and HTLV-1 associated uveitis (HU). A nucleic acid capable of expressing the gp46 antigen of HTLV-1 and a nucleic acid capable of expressing the Tax antigen of HTLV-1 may be encapsulated in the same lipid particle or different lipid particles, and preferably encapsulated separately in different lipid particles.

Using the particle of the present invention, the gp46 antigen and Tax antigen of HTLV-1 can be expressed in vivo or in vitro. More specifically, a lipid particle encapsulating a nucleic acid capable of expressing the gp46 antigen of HTLV-1 and a lipid particle encapsulating a nucleic acid capable of expressing the Tax antigen of HTLV-1 may be administered to a subject. Accordingly, the present invention provides a method for expressing the gp46 antigen and Tax antigen of HTLV-1 in vitro, comprising introducing a composition containing the particle into a cell. The present invention also provides a method for expressing the gp46 antigen and Tax antigen of HTLV-1 in vivo, comprising administering a composition containing the particle to a mammal. An immune response to HTLV-1 can be induced by expressing the gp46 antigen and Tax antigen of HTLV-1 in vivo. As a result, HTLV-1 infection can be prevented and/or treated. Accordingly, the present invention provides a method for inducing an immune response to HTLV-1, comprising administering a composition containing the particle to a mammal. The present invention also provides a method for preventing and/or treating HTLV-1 infection, comprising administering a composition containing the particle to a mammal. The present invention comprises a kit comprising both of the lipid particle encapsulating a nucleic acid capable of expressing the gp46 antigen of HTLV-1 and the lipid particle encapsulating a nucleic acid capable of expressing the Tax antigen of HTLV-1.

The particle of the present invention can be used as a medical drug or a laboratory reagent. The particle of the present invention is usually added to a carrier such as water, buffer and saline, and then, the mixture (composition) can be introduced into a cell (in vitro) or administered to a mammal (in vivo). When the particle is administered to a mammal, a pharmaceutically acceptable carrier (for example, saline) is preferably used as a carrier. The particle of the present invention may be mixed with a base such as fat, fatty oil, lanolin, Vaseline, paraffin, wax, resin, plastic, glycol, higher alcohol, glycerin, water, emulsifier and suspending agent to produce a dosage form such as a cream, a paste, an ointment, a gel and a lotion.

The particle of the present invention may be orally or parenterally (such as intramuscular administration, intravenous administration, intrarectal administration, transdermal administration, transmucosal administration, subcutaneous administration or intradermal administration) administered to a mammal such as a human, a mouse, a rat, a hamster, a guinea pig, a rabbit, a pig, a monkey, a cat, a dog, a horse, a goat, a sheep, and a cow.

The particle of the present invention is administered to a human at a dose of, for example, about 0.001 to 1 mg, preferably 0.01 to 0.2 mg per time per adult on a weight basis of mRNA, once or several times, preferably by intramuscular injection, subcutaneous injection, intradermal injection, intravenous drip or intravenous injection. The dose and frequency of administration herein can be appropriately varied depending on the type of disease, symptom, age, and administration method.

When the particle of the present invention is used as an experimental reagent, the particle is introduced into a cell in which the gp46 antigen and Tax antigen of HTLV-1 are desirably expressed (for example, HEK293 cell and a cell derived therefrom (HEK293T cell, FreeStyle 293 cell, Expi293 cell), CHO cell, C2C12 mouse myoblast, immortalized mouse dendritic cell (MutuDC1940)). In this manner, the gp46 antigen and Tax antigen of HTLV-1 can be expressed in vitro.

Expression of the gp46 antigen and Tax antigen of HTLV-1 can be analyzed by detecting the gp46 antigen protein and Tax antigen protein of HTLV-1 in a sample by western blotting or detecting a peptide fragment specific to the gp46 antigen and Tax antigen of HTLV-1 by mass spectrometry.

In the present invention, “treatment” refers to symptomatic recovery, remission, symptomatic relief and/or delaying the exacerbation of a disease in a patient having an infection caused by, e.g., a virus or bacterium, or a disease (for example, precancer lesion and cancer) caused by the infection.

In the present invention, “prevention” refers to reducing an incidence rate of a disease due to an infection with, e.g., a virus or bacterium. The prevention includes reducing the risk of progression of a disease due to an infection with, e.g., a virus or a bacterium, or reducing exacerbation of a disease. Since the particle of the present invention induces a protective immune response, the particle exerts an effect on the prevention and/or treatment of a disease as mentioned above.

It is expected that the particle of the present invention is used as drugs for prevent the onset of and treating disease caused by HTLV-1 in HTLV-1 infected persons. Examples of the disease caused by HTLV-1 include adult T cell leukemia/lymphoma (ATLL), HTLV-1 associated myelopathy (HAM) and HTLV-1 uveitis (HU).

### EXAMPLES

Now, the present invention will be more specifically described by way of Examples. Note that, these Examples are just for explaining the present invention and thus will not be construed as limiting the range of the present invention.

In Examples, the following brevity codes are sometimes used.

SU gp46: represents surface-unit gp46 of HTLV-1 envelope protein.

gp46-Fib: represents a fusion protein of gp46 and the C-terminal region of Fibritin.

dgp62-Fib: represents a fusion protein of a protein, which is obtained by removing a transmembrane region and intracellular region from gp62, and the C-terminal region of Fibritin.

sec Tax mutant: represents a fusion protein of a mutation-introduced Tax protein and a secretory signal protein.

### [Example 1] Preparation of SU Gp46 mRNA-001

### (1) Preparation of Template DNA for In-Vitro Transcription (IVT) of SU Gp46

For preparing template DNA for use in in-vitro transcription (IVT), SU gp46 DNA was amplified by PCR and then purified. A DNA fragment (SEQ ID NO: 1), which contains a sequence prepared by ligating a T7 promoter sequence, the 5′-UTR sequence of a human β-globin, KOZAK sequence, SU gp46, the 3′-UTR sequence of human β-globin and PolyA sequence, sequentially in this order, was introduced into a plasmid. To Nuclease-free water (547.2 μL) in which the plasmid (8 ng) was dissolved, 10×Buffer for KOD-Plus-Ver. 2 (80 μL, Toyobo Co., Ltd., catalog #KOD-211), 2 mM dNTP mix (80 μL, Toyobo Co., Ltd., catalog #KOD-211), 25 mM MgSO₄ (48 μL, Toyobo Co., Ltd., catalog #KOD-211), a 50 μM sense primer (4.8 μL, SEQ ID NO: 2), a 10 μM antisense primer (24 μL, SEQ ID NO: 3) and KOD Plus polymerase (16 μL, Toyobo Co., Ltd., catalog #KOD-211) were added. The mixture was incubated at 98° C. for one minute and subjected to 20 times of a cycle consisting of a reaction at 98° C. for 5 seconds, a reaction at 55° C. for 15 seconds and a reaction at 68° C. for 90 seconds, and further incubated at 68° C. for one minute to amplify DNA. After completion of the reaction, the template DNA (SEQ ID NO: 4) was purified by Wizard SV Gel and PCR Clean-Up System (Promega catalog #A9281).

(2) Preparation of SU Gp46 mRNA-001 by In-Vitro Transcription

First, 365.1 μg/mL template DNA (4.38 μL) obtained in Example 1-(1), 100 mM CleanCap AG (8 μL, TriLink catalog #N-7113), 100 mM ATP (8 μL, Hongene catalog #R1331), 100 mM GTP (8 μL, Hongene catalog #R2331), 100 mM CTP (8 μL, Hongene catalog #R3331), 100 mM N1-methylpseudoUTP (8 μL, Hongene catalog #R5-027), Nuclease-free water (67.62 μL, Thermo Fisher catalog #AM9937), T7 Transcription 5× buffer (32 μL, Promega catalog #P140X), Enzyme mix and T7 RNA Polymerase (16 μL, Promega catalog #P137X) were mixed and incubated at 37° C. for 4 hours. RQ1 RNase-Free DNase (8 μL, Promega catalog #M6101) was added in the mixture and the resultant mixture was incubated at 37° C. for 15 min. An 8 M LiCl solution (80 μL, Sigma-Aldrich catalog #L7026) was mixed with the mixture and allowed to stand still at −30° C., overnight. After the mixture was centrifuged (4° C., 5200×g, 35 min.), the supernatant was discarded. To the residue, 70% ethanol was added. The mixture was centrifuged (4° C., 5200×g, 10 min.) and then the supernatant was discarded. The residue was air-dried. The obtained residue was dissolved in Nuclease-free water (500 μL) and purified by RNeasy Midi kit (Qiagen catalog #75144) in accordance with the manual attached to the kit. The obtained solution (750 μL), rApid Alkaline Phosphatase (Roche catalog #04 898 141 001) buffer (85 μL), and an enzyme (32 μL) were mixed and incubated at 37° C. for 30 min. and then 75° C. for 2 min. The obtained solution was purified by RNeasy Midi kit (Qiagen catalog #75144) in accordance with the manual attached to the kit to obtain the desired mRNA. The same experimental operation was repeated twice in total. The mRNA solutions obtained were combined to obtain the desired mRNA. The mRNA thus obtained has the sequence of SEQ ID NO: 17.

### [Example 2] Preparation of Gp46-Fib mRNA-002

### (1) Preparation of Template DNA for In-Vitro Transcription (IVT) of Gp46-Fib

To prepare template DNA for use in in-vitro transcription (IVT), gp46-Fib DNA was amplified by PCR and then purified. A DNA fragment (SEQ ID NO: 5), which contains a sequence prepared by ligating a T7 promoter sequence, the 5′-UTR sequence of human β-globin, KOZAK sequence, gp46-Fib, the 3′-UTR sequence of human β-globin and PolyA sequence, sequentially in this order, was introduced into a plasmid. To Nuclease-free water (547.2 μL) in which the plasmid (8 ng) was dissolved, 10×Buffer for KOD-Plus-Ver. 2 (80 μL, Toyobo Co., Ltd., catalog #KOD-211), 2 mM dNTP mix (80 μL, Toyobo Co., Ltd., catalog #KOD-211), 25 mM MgSO₄ (48 μL, Toyobo Co., Ltd., catalog #KOD-211), a 50 μM sense primer (4.8 μL, SEQ ID NO: 2), a 10 μM antisense primer (24 μL, SEQ ID NO: 3) and KOD Plus polymerase (16 μL, Toyobo Co., Ltd., catalog #KOD-211), were added. The mixture was incubated at 98° C. for one minute, and then, subjected to 20 times of a cycle consisting of a reaction at 98° C. for 5 seconds, a reaction at 55° C. for 15 seconds and a reaction at 68° C. for 90 seconds, and further incubated at 68° C. for one minute to amplify DNA. After completion of the reaction, the template DNA (SEQ ID NO: 6) was purified by Wizard SV Gel and PCR Clean-Up System (Promega catalog #A9281).

(2) Preparation of Gp46-Fib mRNA-002 by In-Vitro Transcription

First, 345.3 μg/mL template DNA (4.63 μL) obtained in Example 2-(1), 100 mM CleanCap AG (8 μL, TriLink catalog #N-7113), 100 mM ATP (8 μL, Hongene catalog #R1331), 100 mM GTP (8 μL, Hongene catalog #R2331), 100 mM CTP (8 μL, Hongene catalog #R3331), 100 mM N1-methylpseudoUTP (8 μL, Hongene catalog #R5-027), Nuclease-free water (67.37 μL, Thermo Fisher catalog #AM9937), T7 Transcription 5× buffer (32 μL, Promega catalog #P140X), Enzyme mix and T7 RNA Polymerase (16 μL, Promega catalog #P137X) were mixed and incubated at 37° C. for 4 hours. RQ1 RNase-Free DNase (8 μL, Promega catalog #M6101) was added in the mixture and the resultant mixture was incubated at 37° C. for 15 min. An 8 M LiCl solution (80 μL, Sigma-Aldrich catalog #L7026) was mixed with the mixture and allowed to stand still at −30° C., overnight. After the mixture was centrifuged (4° C., 5200×g, 35 min.), the supernatant was discarded. To the residue, 70% ethanol was added. The mixture was centrifuged (4° C., 5200×g, 10 min.), and then, the supernatant was discarded. The residue was air-dried. The obtained residue was dissolved in Nuclease-free water (500 μL), and then, purified by RNeasy Midi kit (Qiagen catalog #75144) in accordance with the manual attached to the kit. The obtained solution (750 μL), rApid Alkaline Phosphatase (Roche catalog #04 898 141 001) buffer (85 μL), and an enzyme (32 μL) were mixed and incubated at 37° C. for 30 min. and then, 75° C. for 2 min. The obtained solution was purified by RNeasy Midi kit (Qiagen catalog #75144) in accordance with the manual attached to the kit to obtain the desired mRNA. The same experimental operation was repeated twice in total. The mRNA solutions obtained were combined to obtain the desired mRNA. The mRNA thus obtained has the sequence of SEQ ID NO: 18.

### [Example 3] Preparation of Dgp62-Fib mRNA-003

### (1) Preparation of Template DNA for In-Vitro Transcription (IVT) of Dgp62-Fib

To prepare template DNA for use in in-vitro transcription (IVT), dgp62-Fib DNA was amplified by PCR and then purified. A DNA fragment (SEQ ID NO: 7), which contains a sequence prepared by ligating a T7 promoter sequence, the 5′-UTR sequence of human β-globin, KOZAK sequence, dgp62-Fib, the 3′-UTR sequence of human β-globin and PolyA sequence, sequentially in this order, was introduced into a plasmid. To Nuclease-free water (547.2 μL) in which the plasmid (8 ng) was dissolved, 10×Buffer for KOD-Plus-Ver. 2 (80 μL, Toyobo Co., Ltd., catalog #KOD-211), 2 mM dNTP mix (80 μL, Toyobo Co., Ltd., catalog #KOD-211), 25 mM MgSO₄ (48 μL, Toyobo Co., Ltd., catalog #KOD-211), a 50 μM sense primer (4.8 μL, SEQ ID NO: 2), a 10 μM antisense primer (24 μL, SEQ ID NO: 3), and KOD Plus polymerase (16 μL, Toyobo Co., Ltd., catalog #KOD-211), were added. The mixture was incubated at 98° C. for one minute and subjected to 20 times of a cycle consisting of a reaction at 98° C. for 5 seconds, a reaction at 55° C. for 15 seconds and a reaction at 68° C. for 90 seconds and further incubated at 68° C. for one minute to amplify DNA. After completion of the reaction, template DNA (SEQ ID NO: 8) was purified by Wizard SV Gel and PCR Clean-Up System (Promega catalog #A9281).

(2) Preparation of Dgp62-Fib mRNA-003 by In-Vitro Transcription

First, 366.7 μg/mL template DNA (4.36 μL) obtained in Example 3-(1), 100 mM CleanCap AG (8 μL, TriLink catalog #N-7113), 100 mM ATP (8 μL, Hongene catalog #R1331), 100 mM GTP (8 μL, Hongene catalog #R2331), 100 mM CTP (8 μL, Hongene catalog #R3331), 100 mM N1-methylpseudoUTP (8 μL, Hongene catalog #R5-027), Nuclease-free water (67.64 μL, Thermo Fisher catalog #AM9937), T7 Transcription 5× buffer (32 μL, Promega catalog #P140X) and Enzyme mix and T7 RNA Polymerase (16 μL, Promega catalog #P137X) were mixed and incubated at 37° C. for 4 hours. RQ1 RNase-Free DNase (8 μL, Promega catalog #M6101) was added in the mixture and the resultant mixture was incubated at 37° C. for 15 min. An 8 M LiCl solution (80 μL, Sigma-Aldrich catalog #L7026) was mixed with the mixture and allowed to stand still at −30° C., overnight. After the mixture was centrifuged (4° C., 5200×g, 35 min.), the supernatant was discarded. To the residue, 70% ethanol was added and the mixture was centrifuged (4° C., 5200×g, 10 min.). The supernatant was discarded and the residue was air-dried. The obtained residue was dissolved in Nuclease-free water (500 μL) and purified by RNeasy Midi kit (Qiagen catalog #75144) in accordance with the manual attached to the kit. The obtained solution (750 μL), rApid Alkaline Phosphatase (Roche catalog #04 898 141 001) buffer (85 μL), and an enzyme (32 μL) were mixed and incubated at 37° C. for 30 min and then 75° C. for 2 min. The obtained solution was purified by RNeasy Midi kit (Qiagen catalog #75144) in accordance with the manual attached to the kit to obtain the desired mRNA. The same experimental operation was repeated twice in total. The mRNA solutions obtained were combined to obtain the desired mRNA. The mRNA thus obtained has the sequence of SEQ ID NO: 19.

### [Example 4] Preparation of Sec Tax Mutant mRNA-004

### (1) Preparation of Template DNA for In-Vitro Transcription (IVT) of Sec Tax Mutant

To prepare template DNA for use in in-vitro transcription (IVT), sec Tax DNA was amplified by PCR and then purified. A DNA fragment (SEQ ID NO: 9), which contains a sequence prepared by ligating a T7 promoter sequence, the 5′-UTR sequence of human β-globin, KOZAK sequence, sec Tax, the 3′-UTR sequence of human β-globin and PolyA sequence, sequentially in this order, was introduced into a plasmid. To Nuclease-free water (547.2 μL) in which the plasmid (8 ng) was dissolved, 10×Buffer for KOD-Plus-Ver. 2 (80 μL, Toyobo Co., Ltd., catalog #KOD-211), 2 mM dNTP mix (80 μL, Toyobo Co., Ltd., catalog #KOD-211), 25 mM MgSO₄ (48 μL, Toyobo Co., Ltd., catalog #KOD-211), a 50 μM sense primer (4.8 μL, SEQ ID NO: 2), a 10 UM antisense primer (24 μL, SEQ ID NO: 3) and KOD Plus polymerase (16 μL, Toyobo Co., Ltd., catalog #KOD-211), were added. The mixture was incubated at 98° C. for one minute and subjected to 20 times of a cycle consisting of a reaction at 98° C. for 5 seconds, a reaction at 55° C. for 15 seconds and a reaction at 68° C. for 90 seconds, and further incubated at 68° C. for one minute to amplify DNA. After completion of the reaction, template DNA (SEQ ID NO: 10) was purified by Wizard SV Gel and PCR Clean-Up System (Promega catalog #A9281).

(2) Preparation of Sec Tax Mutant mRNA-004 by In-Vitro Transcription

First, 401.1 μg/mL template DNA (3.99 μL) obtained in Example 4-(1), 100 mM CleanCap AG (8 μL, TriLink catalog #N-7113), 100 mM ATP (8 μL, Hongene catalog #R1331), 100 mM GTP (8 μL, Hongene catalog #R2331), 100 mM CTP (8 μL, Hongene catalog #R3331), 100 mM N1-methylpseudoUTP (8 μL, Hongene catalog #R5-027), Nuclease-free water (68.01 μL, Thermo Fisher catalog #AM9937), T7 Transcription 5× buffer (32 μL, Promega catalog #P140X), Enzyme mix and T7 RNA Polymerase (16 μL, Promega catalog #P137X) were mixed and incubated at 37° C. for 4 hours. RQ1 RNase-Free DNase (8 μL, Promega catalog #M6101) was added in the mixture and the resultant mixture was incubated at 37° C. for 15 min. An 8 M LiCl solution (80 μL, Sigma-Aldrich catalog #L7026) was mixed with the mixture and allowed to stand still at −30° C., overnight. After the mixture was centrifuged (4° C., 5200×g, 35 min.), the supernatant was discarded. To the residue, 70% ethanol was added. The mixture was centrifuged (4° C., 5200×g, 10 min.) and then the supernatant was discarded. The residue was air-dried. The obtained residue was dissolved in Nuclease-free water (500 μL) and purified by RNeasy Midi kit (Qiagen catalog #75144) in accordance with the manual attached to the kit. The obtained solution (750 μL), rApid Alkaline Phosphatase (Roche catalog #04 898 141 001) buffer (85 μL), and an enzyme (32 μL) were mixed and incubated at 37° C. for 30 min. and then 75° C. for 2 min. The obtained solution was purified by RNeasy Midi kit (Qiagen catalog #75144) in accordance with the manual attached to the kit to obtain the desired mRNA. The same experimental operation was repeated twice in total. The mRNA solutions obtained were combined to obtain the desired mRNA. The mRNA thus obtained has the sequence of SEQ ID NO: 20.

### [Example 5] Preparation of SU Gp46 mRNA-005

### (1) Preparation of Template DNA for In-Vitro Transcription (IVT) of SU Gp46

Preparation was carried out in the same manner as in Example 1-(1).

(2) Preparation of SU Gp46 mRNA-005 by In-Vitro Transcription

First, 339 μg/mL template DNA (89 μL) obtained in Example 5-(1), 100 mM CleanCap AG (150 μL, TriLink catalog #T-7113), 100 mM ATP (150 μL, Hongene catalog #R1331), 100 mM GTP (150 μL, Hongene catalog #R2331), 100 mM CTP (150 μL, Hongene catalog #R3331), 100 mM N1-methyl-pseudouridine 5′-triphosphate (150 μL, Hongene catalog #R5-027), Nuclease-free water (1261 μL, Qiagen catalog #129114), T7 Transcription 5× buffer (600 μL, Promega catalog #P140X), and Enzyme mix, and T7 RNA Polymerase (300 μL, Promega catalog #P137X) were mixed and incubated at 37° C. for 2 hours. RQ1 RNase-Free DNase (30 μL, Promega catalog #M6101) was added in the mixture and the resultant mixture was incubated at 37° C. for 15 min. An 8 M LiCl solution (1500 μL, Sigma-Aldrich catalog #L7026) was mixed with the mixture and allowed to stand still at −20° C., overnight. After the mixture was centrifuged (4° C., 5250× g, 30 min.), the supernatant was discarded. To the residue, 70% ethanol was added and the mixture was centrifuged (4° C., 5250× g, 10 min.). The supernatant was discarded and the residue was air-dried. The obtained residue was dissolved in Nuclease-free water and purified by RNeasy Maxi kit (Qiagen catalog #75162) in accordance with the manual attached to the kit. The obtained eluate (12 mL, 12.0 mg in terms of UV), Nuclease-free water (120 μL), rApid Alkaline Phosphatase (Roche catalog #04 898 141 001) buffer (1400 μL), and an enzyme (480 μL) were mixed and incubated at 37° C. for 30 min. After completion of incubation at 75° C. for 2 min., an 8M LiCl solution (7000 μL, Sigma-Aldrich catalog #L7026) was added to the mixture. The mixture was allowed to stand still at −20° C., overnight. After the mixture was centrifuged (4° C., 5250×g, 30 min.), the supernatant was discarded. To the residue, 70% ethanol was added. The mixture was centrifuged (4° C., 5250×g, 10 min.) and then the supernatant was discarded. The residue was air-dried. The obtained residue was dissolved in Nuclease-free water (2 mL, 9.9 mg in terms of UV). The mRNA obtained was separated/purified by reversed-phase chromatography (Column: PLRP-S, 4000 Å, 10 μm, 10×200 mm (Agilent), Buffer A: 5% acetonitrile, 400 mM triethylamine acetate (pH7.0), Buffer B: 25% acetonitrile, 400 mM triethylamine acetate (pH7.0), Gradient B %: 27.5% to 35% (20 min.), Flow rate: 5 mL/min, Temperature: 80° C.). The desired fractions were collected and desalted by ultrafiltration (Amicon Ultra-15 (MWCO: 30 kDa)) (3.4 mg in terms of UV).

The mRNA obtained has the sequence of SEQ ID NO: 17. The mRNA was analyzed by LabChip GX Touch Standard RNA Reagent Kit (PerkinElmer catalog #CLS960010) and confirmed to have the desired length.

### [Example 6] Preparation of Sec Tax Mutant mRNA-006

### (1) Preparation of Template DNA for In-Vitro Transcription (IVT) of Sec Tax Mutant

Preparation was carried out in the same manner as in Example 4-(1).

(2) Preparation of Sec Tax Mutant mRNA-006 by In-Vitro Transcription

First, 336 μg/mL template DNA (89 μL) obtained in Example 6-(1), 100 mM CleanCap AG (150 μL, TriLink catalog #T-7113), 100 mM ATP (150 μL, Hongene catalog #R1331), 100 mM GTP (150 μL, Hongene catalog #R2331), 100 mM CTP (150 μL, Hongene catalog #R3331), 100 mM N1-methyl-pseudouridine 5′-triphosphate (150 μL, Hongene catalog #R5-027), Nuclease-free water (1261 μL, Qiagen catalog #129114), T7 Transcription 5× buffer (600 μL, Promega catalog #P140X), and Enzyme mix, and T7 RNA Polymerase (300 μL, Promega catalog #P137X) were mixed and incubated at 37° C. for 2 hours. RQ1 RNase-Free DNase (30 μL, Promega catalog #M6101) was added in the mixture and the resultant mixture was incubated at 37° C. for 15 min. An 8 M LiCl solution (1500 μL, Sigma-Aldrich catalog #L7026) was mixed with the mixture and allowed to stand still at −20° C., overnight. After the mixture was centrifuged (4° C., 5250×g, 30 min.), the supernatant was discarded. To the residue, 70% ethanol was added and the mixture was centrifuged (4° C., 5250× g, 10 min.). The supernatant was discarded and the residue was air-dried. The obtained residue was dissolved in Nuclease-free water, and then, purified by RNeasy Maxi kit (Qiagen catalog #75162) in accordance with the manual attached to the kit. The obtained eluate (12 mL, 12.6 mg in terms of UV), Nuclease-free water (100 μL), rApid Alkaline Phosphatase (Roche catalog #04 898 141 001) buffer (1400 μL) and an enzyme (500 μL) were mixed and incubated at 37° C. for 30 min. After completion of incubation at 75° C. for 2 min., an 8M LiCl solution (7000 μL, Sigma-Aldrich catalog #L7026) was added to the mixture. The mixture was allowed to stand still at −20° C. overnight. After the mixture was centrifuged (4° C., 5250× g, 30 min.), the supernatant was discarded. To the residue, 70% ethanol was added. The mixture was centrifuged (4° C., 5250×g, 10 min.) and then the supernatant was discarded. The residue was air-dried. The obtained residue was dissolved in Nuclease-free water (2 mL, 10.9 mg in terms of UV). The mRNA obtained was separated/purified by reversed-phase chromatography (Column: PLRP-S, 4000 Å, 10 μm, 10×200 mm (Agilent), Buffer A: 5% acetonitrile, 400 mM triethylamine acetate (pH7.0), Buffer B: 25% acetonitrile, 400 mM triethylamine acetate (pH7.0), Gradient B %: 27.5% to 35% (20 min.), Flow rate: 5 mL/min, Temperature: 80° C.). The desired fractions were collected and desalted by ultrafiltration (Amicon Ultra-15 (MWCO: 30 kDa)) (3.7 mg in terms of UV).

The mRNA obtained has the sequence of SEQ ID NO: 20. The mRNA was analyzed by LabChip GX Touch Standard RNA Reagent Kit (PerkinElmer catalog #CLS960010) and confirmed to have the desired length.

### [Example 7] Preparation of Gp46-Fib mRNA-007

### (1) Preparation of Template DNA for In-Vitro Transcription (IVT) of Gp46-Fib

Preparation was carried out in the same manner as in Example 2-(1).

(2) Preparation of Gp46-Fib mRNA-007 by In-Vitro Transcription

First, 397 μg/mL template DNA (63 μL) obtained in Example 7-(1), 100 mM CleanCap AG (50 μL, TriLink catalog #T-7113), 100 mM ATP (50 μL, Hongene catalog #R1331), 100 mM GTP (50 μL, Hongene catalog #R2331), 100 mM CTP (50 μL, Hongene catalog #R3331), 100 mM N1-methyl-pseudouridine 5′-triphosphate (50 μL, Hongene catalog #R5-027), Nuclease-free water (387 μL, Qiagen catalog #129114), T7 Transcription 5× buffer (200 μL, Promega catalog #P140X), and Enzyme mix and T7 RNA Polymerase (100 μL, Promega catalog #P137X) were mixed and incubated at 37° C. for 4 hours. RQ1 RNase-Free DNase (25 μL, Promega catalog #M6101) was added in the mixture and the resultant mixture was incubated at 37° C. for 15 min. An 8 M LiCl solution (500 μL, Sigma-Aldrich catalog #L7026) was mixed with the mixture and allowed to stand still at −20° C., overnight. After the mixture was centrifuged (4° C., 5250× g, 30 min.), the supernatant was discarded. To the residue (pellet), 70% ethanol was added and the mixture was centrifuged (4° C., 5250× g, 30 min.). The supernatant was discarded and the residue (pellet) was air-dried. The obtained residue was dissolved in Nuclease-free water, and thereafter, subjected to purification process performed by the RNeasy Maxi kit (Qiagen catalog #75162) in accordance with the manual attached to the kit. The obtained eluate (3 mL, 3.6 mg in terms of UV), Nuclease-free water (332 μL), rApid Alkaline Phosphatase (Roche catalog #04 898 141 001) buffer (450 μL), and an enzyme (718 μL) were mixed and incubated at 37° C. for 30 min. The mixture was incubated at 75° C. for 2 min., and thereafter, subjected to a purification process performed by RNeasy Maxi kit in accordance with the manual attached to the kit to obtain the desired mRNA (4 mL, 2.8 mg in terms of UV).

The mRNA obtained has the sequence of SEQ ID NO: 18. The mRNA was analyzed by LabChip GX Touch Standard RNA Reagent Kit (PerkinElmer catalog #CLS960010) and confirmed to have the desired length.

### [Example 8] Preparation of Sec Tax Mutant mRNA-008

### (1) Preparation of Template DNA for In-Vitro Transcription (IVT) of Sec Tax Mutant

Preparation was carried out in the same manner as in Example 4-(1).

(2) Preparation of Sec Tax Mutant mRNA-008 by In-Vitro Transcription

First, 391 μg/mL template DNA (64 μL) obtained in Example 8-(1), 100 mM CleanCap AG (50 μL, TriLink catalog #T-7113), 100 mM ATP (50 μL, Hongene catalog #R1331), 100 mM GTP (50 μL, Hongene catalog #R2331), 100 mM CTP (50 μL, Hongene catalog #R3331), 100 mM N1-methyl-pseudouridine 5′-triphosphate (50 μL, Hongene catalog #R5-027), Nuclease-free water (386 μL, Qiagen catalog #129114), T7 Transcription 5× buffer (200 μL, Promega catalog #P140X), Enzyme mix, and T7 RNA Polymerase (100 μL, Promega catalog #P137X) were mixed and incubated at 37° C. for 4 hours. RQ1 RNase-Free DNase (25 μL, Promega catalog #M6101) was added in the mixture and the resultant mixture was incubated at 37° C. for 15 min. An 8 M LiCl solution (500 μL, Sigma-Aldrich catalog #L7026) was mixed with the mixture and allowed to stand still at −20° C., overnight. After the mixture was centrifuged (4° C., 5250× g, 30 min.), the supernatant was discarded. To the residue, 70% ethanol was added and the mixture was centrifuged (4° C., 5250× g, 10 min.). The supernatant was discarded and the residue was air-dried. The obtained residue was dissolved in Nuclease-free water, and thereafter, subjected to a purification process performed by the RNeasy Maxi kit (Qiagen catalog #75162) in accordance with the manual attached to the kit. The obtained eluate (3 mL, 3.5 mg in terms of UV), Nuclease-free water (359 μL), rApid Alkaline Phosphatase (Roche catalog #04 898 141 001) buffer (450 μL) and an enzyme (691 μL) were mixed and incubated at 37° C. for 30 min. The mixture was incubated at 75° C. for 2 min., and thereafter, subjected to a purification process performed by RNeasy Maxi kit in accordance with the manual attached to the kit to obtain the desired mRNA (4 mL, 2.8 mg in terms of UV).

The mRNA obtained has the sequence of SEQ ID NO: 20. The mRNA was analyzed by LabChip GX Touch Standard RNA Reagent Kit (PerkinElmer catalog #CLS960010) and confirmed to have the desired length.

### [Examples 9 to 24] Preparation of Nucleic-Acid Lipid Particles Encapsulating mRNA Molecules Obtained in Examples 1 to 8

(1) Preparation of a Nucleic-Acid Lipid Particle Encapsulating mRNA

Distearoylphosphatidylcholine (1,2-distearoyl-sn-glycero-3-phosphocholine: hereinafter referred to as “DSPC”, NOF CORPORATION), cholesterol (hereinafter referred to as “Chol”, Sigma-Aldrich, Inc.), (7R,9Z,26Z,29R)-18-({[3-(dimethylamino)propoxy]carbonyl}oxy) pentatriaconta-9,26-diene-7,29-diyl diacetate (compound disclosed in Example 23 of WO2015/005253) (hereinafter referred to as “LP1”) or (7R,9Z)-18-({[3-(dimethylamino)propyloxy]carbonyl}oxy)octacosa-9-en-7-yl acetate (compound disclosed in Example 28 of WO2015/005253) (hereinafter referred to as “LP2”), and 1,2-dimyristoyl-sn-glycerol methoxypolyethylene glycol (hereinafter referred to as “PEG-DMG”, NOF CORPORATION, SUNBRIGHT GM-020, a molecular weight of polyethylene glycol: about 2000) were dissolved in ethanol in accordance with the molar ratios listed in Table 1 and so as to have a total lipid concentration of 5 mM.

On the other hand, the mRNA molecules obtained in Examples 1 to 8 were diluted with a citrate buffer (20 mM Citrate Buffer, pH4.0) to prepare dilution solutions.

The lipid solution prepared above and each of the mRNA solutions were mixed in micro-channels so as to have the total lipid weight ratio to the mRNA listed in Table 1 and the volume ratio thereof of 1:3 by use of NanoAssemblr BenchTop (Precision Nanosystems Inc.) to obtain coarse dispersions of nucleic-acid lipid particles. The dispersions of the nucleic-acid lipid particles were dialyzed against about 25 to 50× buffer for 12 to 18 hours (Float-A-Lyzer G2, MWCO: 1,000 kD, Spectra/Por) to remove ethanol. In this manner, purified dispersions of nucleic-acid lipid particles encapsulating mRNA were obtained.

Note that, LP1 was synthesized in accordance with the method disclosed in Example 23 of WO2015/005253; whereas, LP2 in accordance with the method disclosed in Example 28 of WO2015/005253.

(2) Property Evaluation of Nucleic-Acid Lipid Particle Encapsulating mRNA

The dispersions containing the nucleic-acid lipid particles prepared in the above step (1) were subjected to property evaluation. Methods for evaluating individual properties will be described below.

(2-1) Encapsulation Rate of mRNA

The encapsulation rate of mRNA was determined by Quant-iT RiboGreen RNA Assay kit (Invitrogen) in accordance with the package insert.

More specifically, mRNA in each of the dispersions of nucleic-acid lipid particles was measured in the presence and absence of 0.015% Triton X-100 surfactant. The encapsulation rate was calculated in accordance with the following formula:

{([mRNA amount in the presence of surfactant]−[mRNA amount in the absence of surfactant)/[mRNA amount in the presence of surfactant}×100(%)

(2-2) the Ratio of mRNA and Lipid

The amounts of mRNA in the dispersions of nucleic-acid lipid particles were measured in any one of the following methods.

A nucleic-acid lipid particle dispersion was diluted with 1.0% Triton X-100 and subjected to measurement by reversed-phase chromatography (System: Agilent 1260 series, Column: Bioshell A400 Protein C4 (10 cm×4.6 mm, 3.4 μm) (SUPELCO), Buffer A: 0.1 M triethylamine acetate (pH7.0), Buffer B: acetonitrile, (B %): 5-50% (0-15 min.), Flow Rate: 1 mL/min, Temperature: 70° C., Detection: 260 nm).

The nucleic-acid lipid particle dispersion was diluted/dissolved with 90% methanol and subjected to measurement of mRNA amount in nucleic-acid lipid particles by a UV-visible spectrophotometer (manufactured by PerkinElmer Co., Ltd., LAMBDA (trademark) 465). The concentration of mRNA was calculated in accordance with the following formula:

{[Absorbance at 260 nm]−[Absorbance at 350 nm]}×40×dilution ratio(μg/mL)

The amount of lipid contained in each of the nucleic-acid lipid particle dispersions was determined by reversed-phase chromatography (System: DIONEX UltiMate 3000, Column: XSelect CSH C18 (130 Å, 3.5 μm, 3.0 mm×150 mm) (Waters catalog #186005263), Buffer A: 0.2% formic acid, Buffer B: 0.2% formic acid, methanol, (B %): 75-100% (0-6 min.), 100% (6-15 min.), Flow Rate: 0.45 mL/min, Temperature: 50° C., Detection: Corona CAD (Charged Aerosol Detector)).

The weight ratio of the total lipid to mRNA was calculated in accordance with the following formula:

[Total lipid concentration]/[mRNA concentration](wt/wt)

### (2-3) Average Particle Size

The particle sizes of the nucleic-acid lipid particles were measured by Zeta Potential/Particle Sizer NICOMP™ 380ZLS (PARTICLE SIZING SYSTEMS). The average particle size in Tables represents a volume average particle size. A deviation is represented by “± or less”

The property evaluations are listed in Table 2.

TABLE 1

| | | | | | | Lipids/ |
|---|---|---|---|---|---|---|
| Example | | | | | PEG- | mRNA |
| No. | mRNA | DSPC | Chol | LP | DMG | (wt/wt) |
| Example 9 | Example 1 | 12.5% | 41% | 45% (LP1) | 1.5% | 20 |
| Example 10 | Example 2 | 12.5% | 41% | 45% (LP1) | 1.5% | 20 |
| Example 11 | Example 3 | 12.5% | 41% | 45% (LP1) | 1.5% | 20 |
| Example 12 | Example 4 | 12.5% | 41% | 45% (LP1) | 1.5% | 20 |
| Example 13 | Example 5 | 12.5% | 41% | 45% (LP1) | 1.5% | 20 |
| Example 14 | Example 6 | 12.5% | 41% | 45% (LP1) | 1.5% | 20 |
| Example 15 | Example 7 | 17.5% | 26% | 55% (LP2) | 1.5% | 20 |
| Example 16 | Example 8 | 17.5% | 26% | 55%(LP2) | 1.5% | 20 |
| Example 17 | Example 7 | 22.5% | 21% | 55% (LP2) | 1.5% | 20 |
| Example 18 | Example 8 | 22.5% | 21% | 55% (LP2) | 1.5% | 20 |
| Example 19 | Example 7 | 17.5% | 21% | 60% (LP2) | 1.5% | 20 |
| Example 20 | Example 8 | 17.5% | 21% | 60% (LP2) | 1.5% | 20 |
| Example 21 | Example 7 | 22.5% | 16% | 60% (LP2) | 1.5% | 20 |
| Example 22 | Example 8 | 22.5% | 16% | 60% (LP2) | 1.5% | 20 |
| Example 23 | Example 7 | 12.5% | 41% | 45% (LP1) | 1.5% | 20 |
| Example 24 | Example 8 | 12.5% | 41% | 45% (LP1) | 1.5% | 20 |

TABLE 2

| Example No. | Encapsulation rate (%) | Particle size (nm) |
|---|---|---|
| Example 9 | 97% | 126 ± 39 |
| Example 10 | 95% | 138 ± 39 |
| Example 11 | 96% | 133 ± 49 |
| Example 12 | 96% | 124 ± 35 |
| Example 13 | 97% | 111 ± 37 |
| Example 14 | 98% | 108 ± 24 |
| Example 15 | 99% | 135 ± 41 |
| Example 16 | 99% | 133 ± 18 |
| Example 17 | 97% | 138 ± 17 |
| Example 18 | 97% | 133 ± 35 |
| Example 19 | 99% | 123 ± 48 |
| Example 20 | 98% | 123 ± 20 |
| Example 21 | 96% | 127 ± 42 |
| Example 22 | 96% | 129 ± 45 |
| Example 23 | 99% | 133 ± 49 |
| Example 24 | 99% | 137 ± 33 |

From the above results, it was found that 95% or more of the mRNA are encapsulated in the nucleic-acid lipid particles, and that the nucleic-acid lipid particles have an average particle size of about 100 nm to about 140 nm.

### Experimental Example 1

### Construction of Plasmid for LTR Reporter Assay

DNA fragments encoding a HTLV-1 Tax wild type (Uniprot Accession Number: P03409), a Tax mutant, and a secretory Tax mutant, which was prepared by adding an IgE secretory signal to the Tax mutant, were synthesized by an outsourcing company, Eurofin. Each of the DNA fragments was cloned by inserting it into a NheI/NotI site of pcDNA3.1+vector (Thermo Scientific Inc.). DNA fragments encoding HIV-1 LTR and HTLV-1 LTR were synthesized by an outsourcing company, Eurofin. Each of the DNA fragments was cloned by inserting it into a KpnI/XhoI site of pGL4.17 vector (Promega).

### HIV-1 LTR and HTLV-1 LTR Reporter Assay

A plasmid (350 ng) expressing the Tax wild type, Tax mutant, or secretory Tax mutant, an HIV-1 LTR reporter plasmid or a HTLV-1 LTR reporter plasmid (100 ng), and 50 ng of pRG-RK plasmid (Promega) for use in the correction of transfection efficiency were prepared. HEK293T cells were transfected with the plasmids (500 ng in total) by use of transIT-LT1 gene introduction reagent (Takara). Fouty-eight hours after completion of transfection, the expression level of firefly luciferase and the expression level of Renilla luciferase were measured by Dual-Glo (registered trademark) Luciferase Assay System (Promega). The ratio of firefly luciferase expression level/Renilla luciferase expression level was calculated to evaluate the transcriptional activities of HIV-1 LTR and HTLV-1 LTR.

### Experimental Example 2

### Administration of Lipid Particles (Example 12) to C3H Mice

C3H/HeJJc1 mice were obtained from CLEA Japan, Inc and acclimatized. To sterilized musculus triceps femoris of mice under anesthesia, lipid particles (Example 12) were administered at a dose of 5 μg mRNA/20 μL/mouse at intervals of 2 weeks. The first administration was made to the right leg and the second administration to the left leg. The concentration of lipid particles was controlled with a buffer and the buffer was administered to a negative control group.

### Preparation of the Serum and Splenocytes

Two weeks after the first administration of the lipid particles and one week after the second administration, a blood sample was taken from the mice under anesthesia. The blood samples were each allowed to stand still at normal temperature for one hour or more and centrifuged at 3000 RPM for 10 min and collected. The spleen was excised from each of the dead mice by exsanguination under anesthesia. The spleen was ground by the mesh of Cell Strainer (FALCON) and the gasket of a syringe (TERUMO) and added in RPMI 1640 (Nacalai Tesque) to prepare a single-cell suspension. The cells were centrifugally collected. The supernatant was discarded and hemolysis (red blood cell lysis) was carried out with DB Pharm Lyse Lysing buffer (BECTON DICKINSON). The red blood cells lysed were centrifuged and resuspended with RPMI 1640. The cell suspension was passed through mini Cell Strainers (HIGH-TECH CORPORATION, Cat. HT-AMS-14002). After centrifugation, the supernatant was discarded. The cells were resuspended and the density of the cells was measured. After centrifugation, the supernatant was discarded. The density of the cells was adjusted with a cell culture solution (RPMI 1640 containing 10% Fetal Bovine Serum (inactivated and passed through a filter, HyClone), 1% Penicillin-Streptomycin Mixed Solution (Nacalai Tesque), 1% Sodium Pyruvate (gibco), 1% MEM Non-Essential Amino Acids (gibco), 1% HEPES Buffer Solution (gibco) and 1% StemSure Monothioglycerol Solution (FUJIFILM)). Note that, the centrifugation was carried out at 1500 RPM at 4° C. for 5 min.

### Level of Tax-Specific CTL Induction

The mouse splenocytes (3×10⁶ cells) prepared were centrifuged and the supernatant was discarded. A step of resuspending the cells in 1× Dulbecco's Phosphate Buffered Saline (gibco), centrifuging the resuspension and discarding the supernatant was repeated twice. Thereafter, TruStain FcX Antibody (BioLegend) diluted 20 fold with 1×PBS was added to the cells and the mixture was allowed to stand still at normal temperature for 5 min. Five μL of H-2D^{K} HTLV-1 Tax38-46 Tetramer-ARLHTHALL-PE (MBL) was added to each sample. The samples were incubated at 37° C. for 15 min in a 5% CO₂. APC/Fire (trademark) 750 anti-mouse CD3 Antibody (BioLegend) and Anti-CD8 (Mouse) mAb-FITC (MBL), which were diluted 100 fold on ice, were added to the samples, which were allowed to stand still in a dark place for 30 min. The samples were centrifuged and the supernatant was discarded. The cells were washed twice with 1×PBS. Finally, the cells were resuspended with 400 μL of PBS. The cells labeled with FACSVerse (BD) were detected and analyzed by FlowJo (confirmation). Note that, the centrifugation was carried out at 1500 RPM at 4° C. for 5 min.

### Anti-Tax Antibody Titer in Mouse Blood

For using Tax (MYBiosource) recombinant protein as an immobilized antigen, it was added in the wells of a 96-well flat bottom plate and allowed to stand still at 4° C., overnight. A dilution series for a standard curve was prepared by 3-fold serial dilution of Mouse IgG (SouthernBiotech) from the highest concentration of 0.25 μg/mL to obtain 7-stepwise dilution solutions. Individual wells were washed and a blocking solution was added to the wells and allowed to stand still at normal temperature for one hour. The serum samples were prepared with a blocking solution by 4-fold serial dilution from the highest concentration of a 100-fold dilution sample to obtain 7-stepwise dilution solutions. The plate treated with the blocking solution was washed, and a diluted sample was added in the wells of the plate and allowed to stand still at normal temperature for one hour. A detection antibody, Goat Anti-Mouse IgG, and Human ads-HRP (SouthernBiotech) were diluted 3000 fold with the blocking solution and added in the washed wells. One hour later, the plate was washed and then TMB Microwell Peroxidase Substrate System (seracare) was added in the wells. The plate was allowed to stand still for 2 to 3 minutes. The reaction was terminated with TMB Stop Solution (KPL, Cat. 51500-0021). Absorbance was measured at 540 nm and 450 nm by a plate reader. An anti-gp46 antibody titer and anti-Tax antibody titer were calculated by using the corrected absorbance, which was obtained by subtracting the absorbance at 540 nm from the absorbance at 450 nm, in analysis. Note that, as the blocking solution, 1×Dulbecco's Phosphate Buffered Saline (gibco) containing 1% Bovine Serum Albumin (Sigma) and 0.05% Tween (BIO-RAD) was used. Washing was carried out three times with 1×Dulbecco's Phosphate Buffered Saline ((10×) gibco) containing 0.05% Tween.

### Experimental Examples 3 to 5

### Administration to Cynomolgus Monkey

To the upper arms of monkeys, the lipid particles of Example 13 and Example 14 were administered once per two weeks, in total 4 times. The first administration was made to the right upper arm and the following administrations were made alternately to the left and right arms. The lipid particles of Example 13 and Example 14 each contained an equal amount of 25 μg mRNA and the lipid particles were administered at a dose of 50 μg mRNA/200 μL/body per time.

### Experimental Example 3

### Anti Gp46 and Tax Antibody Titer in Monkey Blood

A recombinant gp46 protein (RayBiotech) and a recombination Tax protein (MY Biosource) were added in the wells of 96-well plates and allowed to stand still at 4° C. overnight to immobilize them. Thereafter, the plates were washed by a plate washer (three times with a PBS wash solution containing 0.05% Tween 20) and treated with a blocking solution (PBS containing 1% BSA and 0.05% Tween 20) to perform blocking. The monkey plasma sample dilution series was prepared with a blocking solution by 4-fold serial dilution from the highest concentration of a 100-fold dilution sample to obtain 7-stepwise dilution solutions. A dilution series for a standard curve for monkey IgG concentration was prepared by 3-fold serial dilution of a Monkey IgG solution (provided by a manufacturer) from the highest concentration of 0.25 μg/mL with the blocking solution to obtain 8-stepwise dilution solutions. The sample dilution solution and the dilution solution for the standard curve were added and allowed to stand still at room temperature for one hour. Washing was carried out by a plate washer. As a detection antibody, HRP-labeled anti-monkey IgG antibody (Sigma-Aldrich) was diluted 4000 fold with the blocking solution, added in the wells of the plates and allowed to stand still at room temperature for one hour. After washing was carried out by a plate washer, TMB Microwell Peroxidase Substrate System (SERACARE Life Sciences) was added and allowed to stand still for 10 min. As the reaction termination solution, TMB Stop Solution (SERACARE Life Sciences) was used. Absorbance was measured at a wavelength of 450 nm and a reference wavelength of 540 nm by a plate reader. The corrected absorbance (Delta), which was obtained by subtracting the absorbance at 540 nm from the absorbance at 450 nm, was used in analysis. A calibration curve was prepared based on the monkey IgG concentration in the standard curve and Delta, by use of Nonlinear Regression: 4 Parameters. Based on the calibration curve, the dilution rates of measurement samples and Delta, the anti-gp46 and Tax antibody concentration of the samples were calculated.

### Experimental Example 4

### Cell-Mediated Immune Response Specific to Gp46 and Tax in Monkeys

PBMCs, which were isolated from monkey peripheral blood with Ficoll (Cytiva), were suspended in RPMI Complete culture medium (10% FBS [Sigma-Aldrich], 1% PS [Penicilin-Streptomycin Mixed Solution, Nacalai Tesque], 1 mM Sodium Pyruvate [Thermo Fisher Scientific], 10 mM HEPES [Thermo Fisher Scientific], 1×StemSure [manufactured by FUJIFILM Wako Pure Chemical Corporation], and 1×MEM Non-Essential Amino Acids Solution [Thermo Fisher Scientific]) so as to have a density of 2×10⁶ cells/mL, and seeded in the wells of IFN-γ ELISpot plate (MABTech) at a rate of 100 μL/well. Thereafter, Tax epitope peptide pool (Eurofins), which was prepared with RPMI Complete culture medium to have a final concentration of 0.1% (v/v) or gp46 recombinant protein (RayBiotech), which was prepared to have a final concentration of 1 μg/mL, was added at a rate of 100 μL/well and cultured at 37° C. in a 5% CO₂ condition for 48 hours. Antigen-specific IFN-γ-producing cells were detected by Monkey IFN-γ ELISpot^{PLUS} kit (HRP) (MABTech). The number of the antigen-specific IFN-γ producing cells was counted by ELISPOT analyzer (CTL).

### Experimental Example 5

### Anti-HTLV-1 Neutralization Activity in Monkey Blood

Using Spin column based Antibody Purification Kit (cosmo bio), total IgG was purified from the monkey plasma collected/prepared two weeks after the 4th administration. To YT #1 cell line of HTLV-1 infected cells, purified IgG was added, and then, HTLV-1 uninfected cell line, CEM cells, were mixed at a predetermined ratio of the CEM cells to the YT #1 cells of 1:1. The serum of a patient was added to the co-culture system and cultured overnight. Thereafter, the rate of syncytia formed was measured by microscopic visualization.

### Experimental Example 6

### Total IgG Antibody Titer Specific to Gp46 in Mouse Blood

For using a gp46 (Ray Biotech Inc.) recombinant protein as an immobilized antigen, the protein was added in the wells of a 96-well flat bottom plate and allowed to stand still at 4° C., overnight. A dilution series for a standard curve was prepared by 3-fold serial dilution of Mouse IgG (SouthernBiotech) from the highest concentration of 0.25 μg/mL to obtain 7-stepwise dilution solutions. Individual wells were washed and a blocking solution was added to the wells and allowed to stand still at normal temperature for one hour. The serum samples were prepared with a blocking solution by 4-fold serial dilution from the highest concentration of a 100-fold dilution sample to obtain 7-stepwise dilution solutions. The plate treated with the blocking solution was washed, and a diluted sample was added in the wells of the plate and allowed to stand still at normal temperature for one hour. Detection antibody, Goat Anti-Mouse IgG, and Human ads-HRP (SouthernBiotech) were diluted 3000 fold with the blocking solution and added in the washed wells. One hour later, the wells were washed, and then, TMB Microwell Peroxidase Substrate System (seracare) was added in the wells and allowed to stand still for 2 to 3 minutes. The reaction was terminated with TMB Stop Solution (KPL, Cat. 51500-0021). Absorbance was measured at 540 nm and 450 nm by a plate reader. An anti-gp46 antibody titer and anti-Tax antibody titer were calculated by using the corrected absorbance, which was obtained by subtracting the absorbance at 540 nm from the absorbance at 450 nm, in analysis. Note that, the blocking solution was 1×Dulbecco's Phosphate Buffered Saline (gibco) containing 1% Bovine Serum Albumin (Sigma) and 0.05% Tween (BIO-RAD). Washing was carried out three times with 1×Dulbecco's Phosphate Buffered Saline ((10×) gibco) containing 0.05% Tween.

### Experimental Example 7

### Cell-Mediated Immune Response Specific to Gp46 and Tax in Mice

The mouse splenocytes prepared were seeded in the wells of a 96-well U-bottom plate (FALCON) at a density of 10⁶ cells/well. Then, the mouse splenocytes were stimulated with gp46 and Tax pooled peptides (Eurofins) controlled so as to have a final concentration of 10 μg/mL, at 37° C. in a 5% CO₂ (atmosphere). Twenty-four hours later, the culture supernatant was recovered, and the production amounts of IFN-γ (R&D Systems) and IL-2 (R&D Systems) were measured in accordance with the protocol of the kits.

### Experimental Example 8

### Analysis for Protein Expression

The lipid particles of Examples 9, 10, 11 and 12 each were added to CHO—S cells (Thermo Fisher Scientific) such that the concentration of mRNA in the culture medium became 3 μg/mL. Seventy-two hours after the addition, the culture supernatant and a cell pellet were recovered. To the cell pellet, M-PER (trademark) Tissue Protein Extraction Reagent (Thermo Scientific) was added. The mixture was sufficiently stirred by a vortex and allowed to stand still at normal temperature for 10 min. The mixture was again stirred by a vortex and centrifuged (3000 RPM, 4° C., 10 min.). Thereafter, a cell lysate was collected. The gp46 protein in the culture supernatant and in the cell lysate was detected by Western blotting.

### (Results of Experimental Example 1)

### Screening of Tax Antigen Design Based on Transcriptional Activities of HIV-1 LTR and HTLV-1 LTR as Indexes

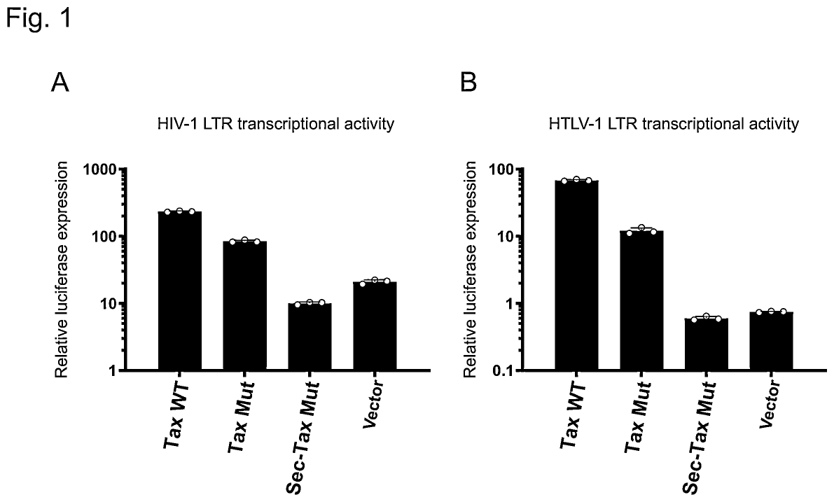

HIV-1 LTR transcriptional activity and HTLV-1 LTR transcriptional activity of the Tax wild type, Tax mutant, and secretory Tax mutant were evaluated (FIG. 1). As a result, it was confirmed that the HIV-1 LTR transcriptional activity and HTLV-1 LTR transcriptional activity of the Tax mutant is lower than those of the Tax wild type. The HIV-1 LTR transcriptional activity and HTLV-1 LTR transcriptional activity of the secretory Tax mutant were equivalent to those of the negative control. From the results, it was suggested that the secretory Tax mutant exhibiting the lowest HIV-1 LTR transcriptional activity and HTLV-1 LTR transcriptional activity is a vaccine-antigen candidate having the lowest carcinogenicity.

### (Results of Experimental Example 2)

### CTL Induction Level and Anti-Tax Antibody Titer in the Blood by the Lipid Particle of Example 12

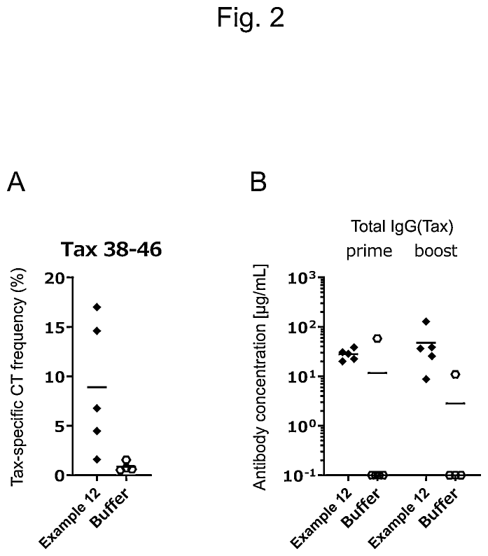

The CTL induction level in C3H mice administered with the lipid particle of Example 12 was evaluated (FIG. 2). As a result, Tax-specific CTL and anti-Tax antibody response in the blood were induced by the lipid particle of Example 12.

### (Results of Experimental Example 3)

### Anti Gp46 and Tax Antibody Titer in Monkey Blood

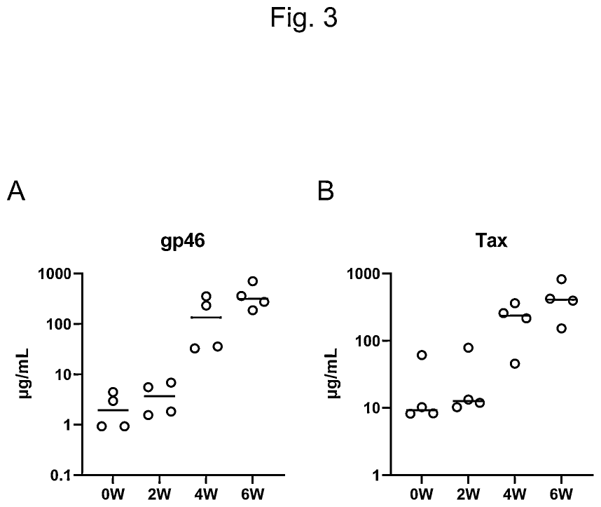

The anti-gp46 antibody response and anti-Tax antibody response in the blood induced by a lipid particle preparation, which was prepared by adding lipid particles of Example 13 and Example 14 in the same amounts, were evaluated (FIG. 3). As a result, the anti-gp46 antibody titer and anti-Tax antibody titer in the blood were high after the 2nd administration (4W) and the 3rd administration (6W) compared to before administration (0W).

### (Results of Experimental Example 4)

### The Level of IFN-γ-Producing Cells Specific to Gp46 and Tax in Monkey PBMCs

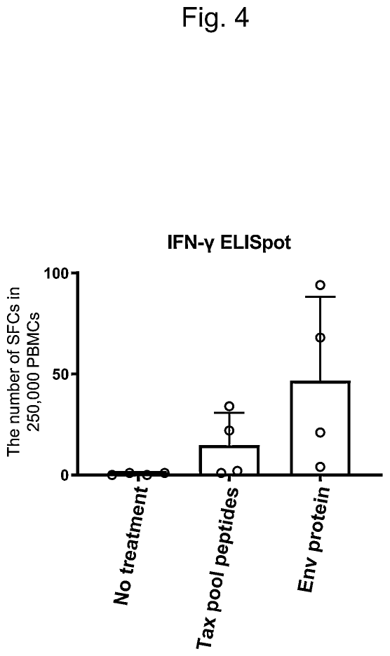

The anti-gp46 antibody response and anti-Tax antibody response in the blood induced by the mixed preparation of the lipid particles of Example 13 and Example 14 were evaluated (FIG. 4). As the results of an examination carried out by use of PBMCs, which were collected/prepared from the blood taken 7 weeks after the 4th administration, it was confirmed that gp46-specific and Tax-specific IFN-γ-producing cells were induced in a group administered with the mixed preparation of the lipid particles of Example 5 and Example 6.

### (Results of Experimental Example 5)

### Anti HTLV-1 Neutralization Activity in Monkey Blood

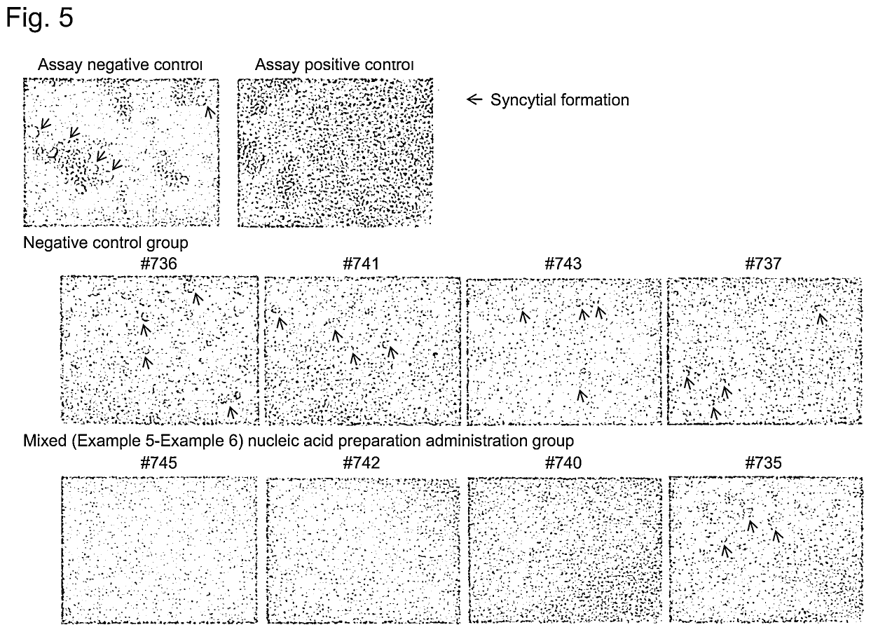

The anti-HTLV-1 neutralization activity in the blood induced by the mixed preparation of the lipid particles of Example 13 and Example 14 was evaluated (FIG. 5). As the results of an examination carried out by use of total IgG in the blood, which was prepared from the blood collected and purified 2 weeks after the 4th administration, the anti-HTLV-1 neutralization activity in the blood was confirmed in 3 of 4 monkeys in a lipid particle administration group, compared to the negative control group.

### (Results of Experimental Example 6)

### Anti Gp46 Antibody Titer in Mouse Blood

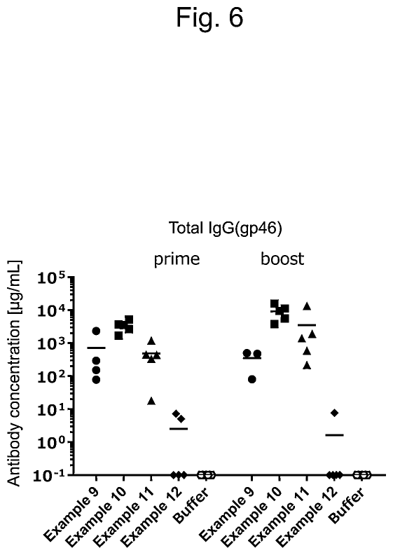

Anti-gp46 antibody response in the blood induced by administration of each of the lipid particles of Example 9, Example 10, Example 11 and Example 12 was evaluated (FIG. 6). As a result, a high anti-gp46 antibody response in the blood was confirmed in the administration groups of the lipid particles of Example 9, Example 10 and Example 11, compared to the negative control group.

### (Results of Experimental Example 7)

### Cell-Mediated Immune Response Specific to Gp46 and Tax in C57BL/6 Mice

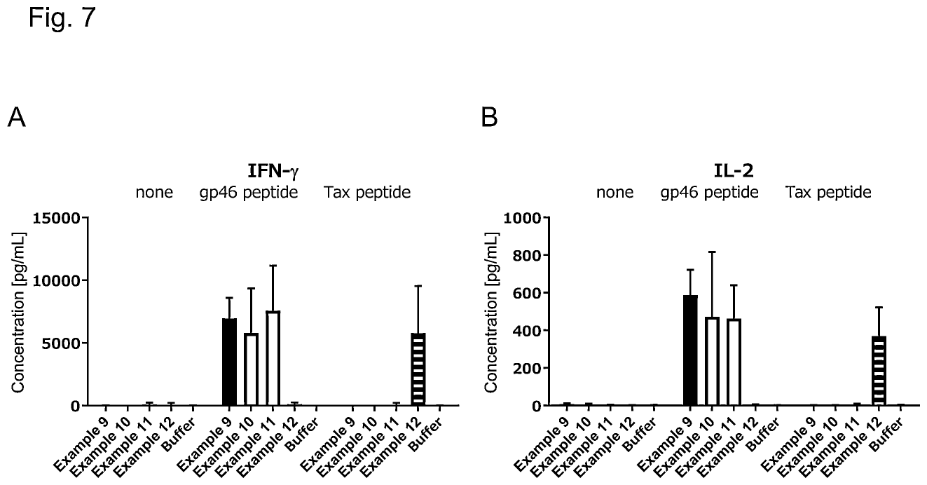

The gp46-specific and IL-2-specific cell-mediated immunity induced by administration of each of the lipid particles of Example 9, Example 10, Example 11 and Example 12 were evaluated based on IFN-γ production level and IL-2 production level as indexes (FIG. 7). As a result, a high-level gp46-specific cell-mediated immune response was confirmed in the lipid particle administration groups of Example 9, Example 10 and Example 11, compared to the negative control group. In the lipid particle administration group of Example 12, a high-level Tax-specific cell-mediated immune response was confirmed, compared to the negative control group.

### (Results of Experimental Example 8)

### Antigen Protein Expression Level by Lipid Particles of Example 9 to Example 12

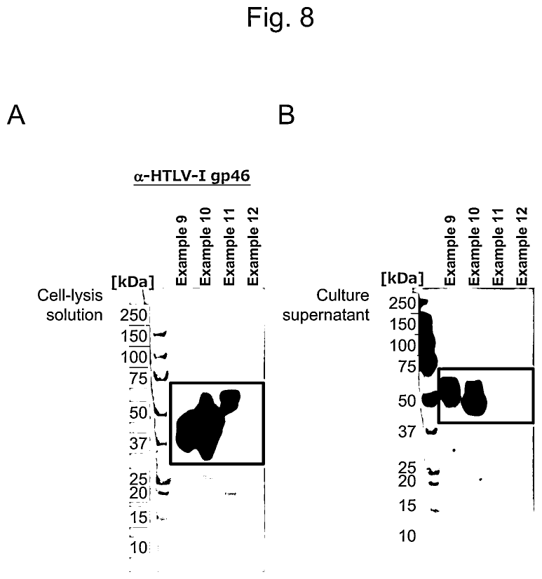

In the culture supernatants of CHO—S cells treated with the lipid particles of Example 9 to Example 12 and cell lysates, the expression levels of antigen proteins expressed by the lipid particles were evaluated (FIG. 8). As a result, it was confirmed that antigen proteins, to which the anti-gp46 antibodies expressed by individual lipid particles bind, are expressed. Note that, the size of the gp46 protein was 39 kDa.

### Examples 25 to 29

Preparation of Gp46-Fib mRNA

### (1) Preparation of Template DNA for In-Vitro Transcription (IVT) of Gp46-Fib

Plasmids were constructed for preparing template DNA for use in in-vitro transcription (IVT). Plasmids were prepared by introducing DNA fragments (SEQ ID NOs: 21 to 25) each containing a sequence prepared by ligating a T7 promoter sequence, a 5′-UTR sequence of human β-globin, KOZAK sequence, a coding region of gp46-Fib, 3′-UTR sequence of human β-globin and a polyA sequence, sequentially in the order (Examples 25 to 29 were carried out by using plasmids prepared by introducing DNA fragments of SEQ ID NOs: 21 to 25, respectively).

### (2) Linearization of Template DNA

In Nuclease-free water (268 μL), in which the plasmid (300 μg) obtained in the above step (1) was dissolved, a 10×NE Buffer 3.1 and BspQI (32 μL, New England Biolabs catalog #R0712L) were added. The mixture was incubated at 50° C. for 16 hours, and then, 80° C. for 20 minutes. To this, ethanol (900 μL) and a 3 mol/L sodium acetate solution (30 μL) were mixed. The mixture was allowed to stand still at −80° C. for 4 hours. Centrifugation (4° C., 15000× g, 30 min.) was carried out and the supernatant was discarded. To the residue, 70% ethanol was added. The suspension was centrifugated (4° C., 15000× g, 10 min.). The supernatant was discarded and the residue was air-dried. The obtained residue was dissolved in Nuclease-free water to prepare a 500 μg/mL solution.

(3) Preparation of Gp46-Fib mRNA by In-Vitro Transcription

The 500 μg/mL template DNA (15 μL) obtained in the above step (2), 100 mM CleanCap AG (15 μL, TriLink catalog #N-7113), 100 mM ATP (15 μL, Hongene catalog #R1331), 100 mM GTP (15 μL, Hongene catalog #R2331), 100 mM 5-Me-CTP (15 μL, Hongene catalog #R3-029), 100 mM 5-Me-UTP (15 μL, Hongene catalog #R5-104), Nuclease-free water (113 μL), 5×IVT buffer (60 μL, 400 mM HEPES-KOH PH7.5, 400 mM DTT, 120 mM MgCl2, 10 mM Spermidine), T7 RNA Polymerase (15 μL, Promega catalog #P407X), RNase Inhibitor (15 μL, Promega catalog #P261X) and Pyrophosphatase (7.5 μL, Hongene catalog #ON-025) were mixed. The mixture was incubated at 37° C. for 4 hours. RQ1 RNase-Free DNase (7.5 μL, Promega catalog #M6101) was added in the mixture and the resultant mixture was incubated at 37° C. for 30 min. To the mixture, a 10 M ammonium acetate solution (100 μL) was added. The mixture was allowed to stand still at −20° C. for 2 hours. After the mixture was centrifuged (4° C., 15000×g, 30 min.), the supernatant was discarded. To the residue, 70% ethanol was added. The suspension was centrifugated (4° C., 15000×g, 10 min.). The supernatant was discarded and the residue was air-dried. The obtained residue was dissolved in Nuclease-free water and purified with NucleoSpin (Macherey-Nagel catalog #740948.50) to obtain the desired mRNA.

The obtained mRNA molecules have the sequences of SEQ ID NOs: 31 to 35, a Cap-1 structure at the 5′-end and 5-methylcytidine and 5-methyluridine in place of cytidine and uridine, respectively. It was confirmed that the mRNA molecules have desired lengths based on the analysis by LabChip GX Touch Standard RNA Reagent Kit (PerkinElmer catalog #CLS960010).

### Examples 30 to 34

Preparation of Nucleic-Acid Lipid Particles Encapsulating mRNA Obtained in Examples 25 to 29
(1) Preparation of Nucleic-Acid Lipid Particle Encapsulating mRNA

The nucleic-acid lipid particles listed in Table 3 were prepared in the same manner as in Examples 9 to 24 except that the mRNA molecules obtained in Examples 25 to 29 were respectively used in place of the mRNA molecules obtained in Examples 1 to 8.

(2) Property Evaluation of Nucleic-Acid Lipid Particle Encapsulating mRNA

The properties of the nucleic-acid lipid particles were evaluated in the same manner as in Examples 9 to 24. The results are listed in Table 4.

TABLE 3

| | | | | | | Lipids/ |
|---|---|---|---|---|---|---|
| Example | | | | | PEG- | mRNA |
| No. | mRNA | DSPC | Chol | LP | DMG | (wt/wt) |
| Example | Example | 17.5% | 21% | 60% | 1.5% | 25 |
| 35 | 25 | | | (LP2) | | |
| Example | Example | 17.5% | 21% | 60% | 1.5% | 25 |
| 36 | 26 | | | (LP2) | | |
| Example | Example | 17.5% | 21% | 60% | 1.5% | 25 |
| 37 | 27 | | | (LP2) | | |
| Example | Example | 17.5% | 21% | 60% | 1.5% | 25 |
| 38 | 28 | | | (LP2) | | |
| Example | Example | 17.5% | 21% | 60% | 1.5% | 25 |
| 39 | 29 | | | (LP2) | | |
| Example | Example | 17.5% | 21% | 60% | 1.5% | 25 |
| 40 | 30 | | | (LP2) | | |

TABLE 4

| Example No. | Encapsulation rate (%) | Particle size (nm) |
|---|---|---|
| Example 35 | 95% | 107 ± 13 |
| Example 36 | 96% | 130 ± 28 |
| Example 37 | 96% | 111 ± 22 |
| Example 38 | 96% | 103 ± 16 |
| Example 39 | 96% | 108 ± 13 |
| Example 40 | 97% | 102 ± 28 |

### (Experimental Example 9)

### Analysis for Expression of Gp46 Protein

The nucleic-acid lipid particles of Examples 30 to 34 were added to CHO—S cells (Thermo Fisher Scientific) such that the mRNA concentration of a medium became 3 μg/mL. As the negative control, a buffer of the same volume as the liquid used in the particles of Examples 35 to 39 was added. Three days after addition of the particles, the culture supernatant was recovered. The gp46 protein in the culture supernatant was detected by diluting the culture supernatant 10× with D-PBS, immobilizing the supernatant onto a 96 half-well plate and applying Enzyme-Linked Immunosorbent Assay (ELISA) using the serum of a mouse administered with the (nucleic acid) lipid particles of Example 10.

### (Results of Experimental Example 9)

### Ability of Nucleic-Acid Lipid Particles of Examples 30 to 34 to Induce Expression of Gp46

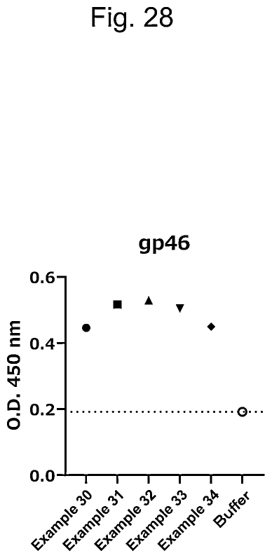

The expression levels of gp46 protein in the culture supernatants of CHO—S cells treated with nucleic-acid lipid particles of Examples 30 to 34, were evaluated. As a result, the CHO—S cells treated with nucleic-acid lipid particles of Examples 30 to 34 expressed gp46 protein (FIG. 28).

### INDUSTRIAL APPLICABILITY

The present invention can be used prevention and/or treatment of infection with HTLV-1.

### [Sequence Listing Free Text]

SEQ ID NO: 1: Nucleotide sequence of template plasmid DNA for IVT of SU gp46

SEQ ID NO: 2: Nucleotide sequence of a sense primer

SEQ ID NO: 3: Nucleotide sequence of an antisense primer

SEQ ID NO: 4: Nucleotide sequence of template DNA for in-vitro transcription (IVT) of SU gp46

SEQ ID NO: 5: Nucleotide sequence of template plasmid DNA for IVT of HTLV-I gp46-Fib

SEQ ID NO: 6: Nucleotide sequence of template DNA for in-vitro transcription (IVT) of gp46-Fib

SEQ ID NO: 7: Nucleotide sequence of template plasmid DNA for IVT of HTLV-I dgp62-Fib

SEQ ID NO: 8: Nucleotide sequence of template DNA for in-vitro transcription (IVT) of dgp62-Fib

SEQ ID NO: 9: Nucleotide sequence of template plasmid DNA for IVT of HTLV-I sec Tax mutant

SEQ ID NO: 10: Nucleotide sequence of template DNA for in-vitro transcription (IVT) of sec Tax mutant

SEQ ID NO: 11: Amino acid sequence of Tax wild type

SEQ ID NO: 12: Amino acid sequence of Tax mutant (T130A/L131S/L319R/L320S)

SEQ ID NO: 13: Amino acid sequence of secretory Tax mutant (T130A/L131S/L319R/L320S)

SEQ ID NO: 14: Amino acid sequence of gp46

SEQ ID NO: 15: Amino acid sequence of gp46-Fib

SEQ ID NO: 16: Amino acid sequence of dgp62-Fib

SEQ ID NO: 17: mRNA of SU gp46

SEQ ID NO: 18: mRNA of gp46-Fib

SEQ ID NO: 19: mRNA of dgp62-Fib

SEQ ID NO: 20: mRNA of sec Tax mutant

SEQ ID NO: 21: nucleotide sequence of the template DNA for gp46-Fib+polyA95

SEQ ID NO: 22: Nucleotide sequence of template DNA for gp46-Fib+polyA80

SEQ ID NO: 23: Nucleotide sequence of template DNA for gp46-Fib+polyA60

SEQ ID NO: 24: Nucleotide sequence of template DNA for gp46-Fib+polyA40

SEQ ID NO: 25: Nucleotide sequence of template DNA for gp46-Fib+polyA20

SEQ ID NO: 26: Nucleotide sequence of template DNA for a sec Tax mutant+polyA95

SEQ ID NO: 27: Nucleotide sequence of template DNA for a sec Tax mutant+polyA80

SEQ ID NO: 28: Nucleotide sequence of template DNA for a sec Tax mutant+polyA60

SEQ ID NO: 29: Nucleotide sequence of template DNA for a sec Tax mutant+polyA40

SEQ ID NO: 30: Nucleotide sequence of template DNA for a sec Tax mutant+polyA20

SEQ ID NO: 31: mRNA sequence of gp46-Fib+polyA95

SEQ ID NO: 32: mRNA sequence of gp46-Fib+polyA80

SEQ ID NO: 33: mRNA sequence of gp46-Fib+polyA60

SEQ ID NO: 34: mRNA sequence of gp46-Fib+polyA40

SEQ ID NO: 35: mRNA sequence of gp46-Fib+polyA20

SEQ ID NO: 36: mRNA sequence of sec Tax mutant+polyA95

SEQ ID NO: 37: mRNA sequence of sec Tax mutant+polyA80

SEQ ID NO: 38: mRNA sequence of sec Tax mutant+polyA60

SEQ ID NO: 39: mRNA sequence of sec Tax mutant+polyA40

SEQ ID NO: 40: mRNA sequence of sec Tax mutant+polyA20

All publications, patents and patent applications cited in the specification are incorporated herein in their entireties by reference.

1. A lipid particle comprising:
a cationic lipid of formula (Ia): 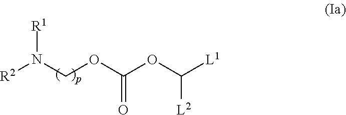
or a pharmaceutically acceptable salt thereof,
wherein
R¹ and R² are each independently a C₁-C₃ alkyl group;
L¹ is a C₁₇-C₁₉ alkenyl group, or a C₁₇-C₁₉ alkenyl group having one or more C₂-C₄ alkanoyloxy groups;
L² is a C₁₀-C₁₉ alkyl group, a C₁₀-C₁₉ alkyl group having one or more C₂-C₄ alkanoyloxy groups, a C₁₀-C₁₉ alkenyl group, or a C₁₀-C₁₉ alkenyl group having one or more C₂-C₄ alkanoyloxy groups; and
p is 3 or 4; and
a nucleic acid encapsulated by the lipid particle, wherein the nucleic acid is capable of expressing a gp46 antigen or a Tax antigen of human T-cell leukemia virus type 1 (HTLV-1).

2. The lipid particle of claim 1, wherein, R¹ and R² both are methyl groups.

3. The lipid particle of claim 1, wherein, p is 3.

4. The lipid particle of claim 1, wherein L¹ is a C₁₇-C₁₉ alkenyl group, or a C₁₇-C₁₉ alkenyl group having one or more acetoxy groups.

5. The lipid particle of claim 1, wherein L² is a C₁₀-C₁₂ alkyl group, a C₁₀-C₁₂ alkyl group having one or more acetoxy groups, a C₁₀-C₁₉ alkenyl group, or a C₁₀-C₁₉ alkenyl group having one or more acetoxy groups.

6. The lipid particle of claim 1, wherein L² is a C₁₀-C₁₂ alkyl group, C₁₀-C₁₂ alkyl group having one or more acetoxy groups, a C₁₇-C₁₉ alkenyl group, or a C₁₇-C₁₉ alkenyl group having one or more acetoxy groups.

7. The lipid particle of claim 1, wherein L¹ is an (R)-11-acetyloxy-cis-8-heptadecenyl group, a cis-8-heptadecenyl group, or an (8Z,11Z)-heptadecadienyl group.

8. The lipid particle of claim 1, wherein L² is a decyl group, a cis-7-decenyl group, a dodecyl group, or an (R)-11-acetyloxy-cis-8-heptadecenyl group.

9. The lipid particle of claim 1, wherein the cationic lipid has structural formula:

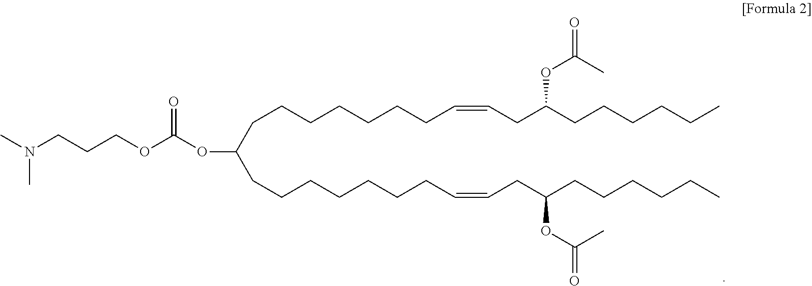 [SMILES: CCCCCC[C@H](C/C=C\CCCCCCCC(CCCCCCC/C=C\C[C@@H](CCCCCC)OC(C)=O)OC(=O)OCCCN(C)C)OC(C)=O]

10. The lipid particle of claim 1, wherein the cationic lipid has structural formula:

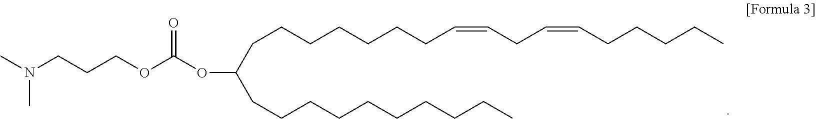 [SMILES: CCCCC/C=C\C/C=C\CCCCCCCC(CCCCCCCCCC)OC(=O)OCCCN(C)C]

11. The lipid particle of claim 1, wherein the cationic lipid has structural formula:

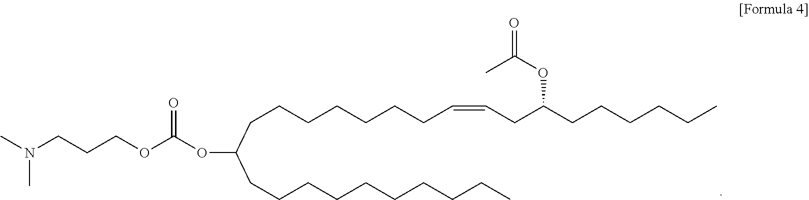 [SMILES: CCCCCCCCCCC(CCCCCCC/C=C\C[C@@H](CCCCCC)OC(C)=O)OC(=O)OCCCN(C)C]

12. The lipid particle of claim 9, wherein the lipid particle further comprises an amphipathic lipid, a sterol, and a PEG lipid.

13. The lipid particle of claim 11, wherein the lipid particle further comprises an amphipathic lipid, a sterol and, a PEG lipid.

14. The lipid particle of claim 12, wherein the amphipathic lipid is at least one of distearoylphosphatidylcholine, dioleoylphosphatidylcholine and dioleoyl phosphatidylethanolamine.

15. The lipid particle of claim 13, wherein the amphipathic lipid is at least one of distearoylphosphatidylcholine, dioleoylphosphatidylcholine, and dioleoyl phosphatidylethanolamine.

16. The lipid particle of claim 12, wherein the sterol is cholesterol.

17. The lipid particle of claim 13, wherein the sterol is cholesterol.

18. The lipid particle of claim 12, wherein the PEG lipid is one or more of 1,2-dimyristoyl-sn-glycerol methoxypolyethylene glycol, and N-[methoxypoly(ethylene glycol)2000]carbamoyl]-1,2-dimyristyloxypropyl-3-amine.

19. The lipid particle of claim 13, wherein the PEG lipid is one or more of 1,2-dimyristoyl-sn-glycerol methoxypolyethylene glycol, and N-[methoxypoly(ethylene glycol)2000]carbamoyl]-1,2-dimyristyloxypropyl-3-amine.

20. The lipid particle of claim 12 comprising: 5 to 25% of the amphipathic lipid, 10 to 55% of the sterol, 40 to 65% of the cationic lipid, and 1 to 5% of the PEG lipid on a molar basis.

21. The lipid particle of claim 20, wherein the amphipathic lipid is 10 to 25%.

22. The lipid particle of claim 12 comprising: 5 to 15% of the amphipathic lipid, 35 to 50% of the sterol, 40 to 55% of the cationic lipid, and 1 to 3% of the PEG lipid on a molar basis.

23. The lipid particle of claim 22, wherein the amphipathic lipid is 10 to 15%, the sterol is 35 to 45%, the cationic lipid is 40 to 50%, and the PEG lipid is 1 to 2.5%.

24. The lipid particle of claim 23, wherein the PEG lipid is 1 to 2%.

25. The lipid particle of claim 13 comprising: 10 to 25% of the amphipathic lipid, 10 to 50% of the sterol, 40 to 65% of the cationic lipid, and 1 to 3% of the PEG lipid on a molar basis.

26. The lipid particle of claim 25, wherein the sterol is 10 to 45%, the cationic lipid is 42.5 to 65%, and the PEG lipid is 1 to 2.5%.

27. The lipid particle of claim 26, wherein the PEG lipid is 1 to 2%.

28. The lipid particle of claim 20, wherein a weight ratio of total lipid to the nucleic acid is from 15 to 30.

29. The lipid particle of claim 28, wherein the weight ratio of total lipid to the nucleic acid is from 15 to 25.

30. The lipid particle of claim 29, wherein the weight ratio of total lipid to the nucleic acid is from 17.5 to 22.5.

31. The lipid particle of claim 1, wherein the gp46 antigen of human T-cell leukemia virus type 1 (HTLV-1) is a fusion protein with an oligomerization domain.

32. The lipid particle of claim 31, wherein the oligomerization domain is fibritin.

33. The lipid particle of claim 31, wherein the gp46 antigen of human T-cell leukemia virus type 1 (HTLV-1) has an amino acid sequence identity of at least 95% with SEQ ID NO: 14.

34. The lipid particle of claim 1, wherein the Tax antigen of human T-cell leukemia virus type 1 (HTLV-1) has at least one mutation selected from the group consisting of T130A, L131S, L319R and L320S relative to SEQ ID NO: 11, and the amino acid sequence consisting of amino acids except the mutant amino acids has an identity of at least 95% with the amino acid sequence of SEQ ID NO: 11.

35. The lipid particle of claim 34, wherein the Tax antigen of human T-cell leukemia virus type 1 (HTLV-1) has mutations of T130A, L131S, L319R and L320S relative to SEQ ID NO: 11, and the amino acid sequence consisting of amino acids except the mutant amino acids has an identity of at least 95% with the amino acid sequence of SEQ ID NO: 11.

36. The lipid particle of claim 1, wherein the Tax antigen of human T-cell leukemia virus type 1 (HTLV-1) is a fusion protein with a signal peptide.

37. The lipid particle of claim 36, wherein the signal peptide is amino acids 1 to 18 of SEQ ID NO: 13.

38. The lipid particle of claim 31, wherein the nucleic acid capable of expressing the gp46 antigen or Tax antigen of human T-cell leukemia virus type 1 (HTLV-1) is an mRNA containing a cap structure (Cap), a 5′-noncoding region (5′-UTR), a coding region of the gp46 antigen or Tax antigen, a 3′-noncoding region (3′-UTR), and poly A tail (polyA).

39. The lipid particle of claim 38, wherein the nucleic acid capable of expressing the gp46 antigen of human T-cell leukemia virus type 1 (HTLV-1) has a nucleotide sequence identity of at least 90% with SEQ ID NO: 17 18, or 20.

40. The lipid particle of claim 38, wherein the sequence of the nucleic acid capable of expressing the Tax antigen of human T-cell leukemia virus type 1 (HTLV-1) has a nucleotide sequence identity of at least 90% with SEQ ID NO: 20.

41. The lipid particle of claim 1, wherein the nucleic acid comprises at least one modified nucleotide.

42. The lipid particle of claim 41, wherein the modified nucleotide comprises at least one of a pyrimidine nucleotide substituted at position 5, a pseudouridine, and pseudouridine substituted at position 1.

43. The lipid particle of claim 41, wherein the modified nucleotide comprises at least one of 5-methylcytidine, 5-methoxyuridine, 5-methyluridine, pseudouridine, and a 1-alkylpseudouridine.

44. The lipid particle of claim 41, wherein the modified nucleotide comprises at least one of 5-methylcytidine, 5-methyluridine, and 1-methylpseudouridine.

45. The lipid particle of claim 1, having an average particle size of 30 to 300 nm.

46. A method of preparing a composition for preventing and/or treating infection with human T-cell leukemia virus type 1 (HTLV-1), comprising: combining the lipid particle of claim 1 and a pharmaceutically acceptable carrier.

47. A composition comprising: a plurality of the lipid particle of claim 1.

48. The composition of claim 47, wherein the composition is used for expressing the gp46 antigen or Tax antigen of human T-cell leukemia virus type 1 (HTLV-1) in vivo or in vitro.

49. The composition of claim 47 comprising: a pharmaceutically acceptable carrier, wherein the composition is a medicament.

50. The composition of claim 49, wherein the composition is used for inducing an immune response to human T-cell leukemia virus type 1 (HTLV-1).

51. The composition according of claim 49, wherein the composition is used for preventing and/or treating infection with human T-cell leukemia virus type 1 (HTLV-1).

52. The composition of claim 49, wherein the composition is used for preventing the onset of and/or treating a disease caused by HTLV-1 selected from the group consisting of adult T cell leukemia/lymphoma (ATLL), HTLV-1 associated myelopathy (HAM) and HTLV-1 uveitis (HU), in an HTLV-1 infected person.

53. A method for expressing a gp46 antigen or Tax antigen of human T-cell leukemia virus type 1 (HTLV-1) in vitro, comprising: introducing the composition of claim 47 into a cell.

54. A method for expressing a gp46 antigen or Tax antigen of human T-cell leukemia virus type 1 (HTLV-1) in vivo, comprising: administering the composition of claim 47 to a mammal.

55. A method of treating a mammal, comprising: administering the composition of claim 49 to the mammal; and inducing an immune response to human T-cell leukemia virus type 1 (HTLV-1).

56. A method for preventing and/or treating infection with human T-cell leukemia virus type 1 (HTLV-1), comprising: administering the composition of claim 49 to a mammal.

57. A method for preventing the onset of and/or treating a disease caused by HTLV-1, comprising: administering the composition of claim 49 to a mammal, wherein the disease is selected from the group consisting of adult T cell leukemia/lymphoma (ATLL), HTLV-1 associated myelopathy (HAM) and HTLV-1 uveitis (HU).
